(19) United States
(12) Patent Application Publication
(10) Pub. No.: US 2020/0390689 A1
(43) Pub. Date: Dec. 17, 2020

(54) CERDULATINIB-CONTAINING TOPICAL SKIN PHARMACEUTICAL COMPOSITIONS AND USES THEREOF

(71) Applicant: Dermavant Sciences GmbH, Basel (CH)

(72) Inventors: Charles Rodney Greenaway EVANS, West Sussex (GB); Cameron Robert STEVENSON, Surrey (GB); Marc Barry BROWN, Hertfordshire (GB)

(21) Appl. No.: 16/960,572

(22) PCT Filed: Jan. 9, 2019
(86) PCT No.: PCT/IB2019/000017

§ 371 Date: Jul. 8, 2020

Related U.S. Application Data
(60) Provisional application No. 62/772,415, filed on Nov. 28, 2018.
(60) Provisional application No. 62/765,133, filed on Aug. 16, 2018.
(60) Provisional application No. 62/686,509, filed on Jun. 18, 2018.
(60) Provisional application No. 62/615,261, filed on Jan. 9, 2018.

(51) Int. Cl. A61K 9/00; A61K 31/506; A61K 47/10; A61K 47/38; A61K 9/06

(52) U.S. Cl. CPC A61K 9/0014; A61K 31/506; A61K 9/06; A61K 47/38; A61K 47/10

(57) ABSTRACT

Embodiments of topical formulations for administering cerdulatinib or a pharmaceutically acceptable salt, hydrate or solvate thereof are disclosed. Embodiments of methods for preparing the topical formulations are also disclosed. The disclosed formulations are suitable for the treatment of dermatologic conditions such as atopic dermatitis.

### FIELD OF THE INVENTION

The present disclosure relates to topical formulations containing cerdulatinib and methods of using the formulations in the treatment of dermatological disorders, for example atopic dermatitis, alopecia areata, vitiligo, and chronic urticaria.

### BACKGROUND OF THE INVENTION

Atopic dermatitis (AD) is clinically defined as a chronic intermittent disease of the skin characterized by intense itch (pruritus) and inflammatory eczematous lesions. It is one of the most common chronic diseases, affecting 10 to 20% of the population in developed countries [Deckers, 2012; Williams, 2008]. AD occurs more commonly in children, affecting 15 to 30% of the pediatric population [Williams, 2006], whereas approximately 10% of adults are affected [Silverberg, 2013]. Among pediatric populations, approximately 60% of patients present in the first year of life [Illi, 2004; Garmhausen, 2013], and about 85% of patients present by age 5 [Bieber, 2008].

Atopic dermatitis is mild to moderate in most patients, with 70% of all patients and 80% of children having mild disease [Ballardini, 2013]. Twenty percent of patients have moderate to severe disease, characterized by clinical features that are chronic and relapsing. Both genetic and environmental factors contribute to the pathogenesis of the disease, which is characterized by defects in the skin barrier and immune system dysregulation [Kuo, 2013; Boguniewicz, 2011]. The skin lesions that result from these defects are painful, and their appearance can cause the patient social and psychological harm [Dalgard, 2015]. Beyond the immediate physical symptoms and psychological manifestations of the AD lesions, the disease has profound secondary effects on the well-being of patients. Specifically, pruritus associated with the disease causes significant discomfort, often leading to sleep deprivation in the patient. Sleeplessness in young patients also negatively affects sleep quality in the parents of the afflicted children.

Despite the high prevalence of AD, there are limited treatment options available for patients. The first-line treatment option for patients with mild to moderate disease is topical corticosteroids. However, many patients are steroid refractory and there are significant long-term safety risks associated with their use [Atherton, 2003]. Topical calcineurin inhibitors, pimecrolimus and tacrolimus, are used as second-line treatment options, but are not effective in many patients. Additionally, product labeling for each of these drugs includes a boxed warning for carcinogenicity risks (as class labeling for topical calcineurin inhibitors) [Elidel, 2014; Protopic, 2012]. Crisaborole, a topical phosphodiesterase 4 (PDE4PDE4) inhibitor has recently been approved for children and adults with mild to moderate atopic dermatitis. Dupilumab, a novel monoclonal antibody (mAb) targeting the IL-4 receptor alpha (IL-4Rα), is approved for the treatment of patients with moderate to severe AD whose disease is not adequately controlled with topical prescription therapies or when those therapies are not advisable. However, dupilumab requires frequent subcutaneous injections and is currently approved for adults. Therefore, a need remains for a topical therapy that is both safe and efficacious for subjects with mild to severe AD.

Atopic dermatitis results from dysregulation of the interplay between keratinocytes, immune cells, and the environment that results in the production of type 2 cytokines; although the precise pathogenesis has not yet been fully elucidated. A hallmark of AD is the marked influx of T lymphocytes within both the dermis and epidermis of lesional skin [Werfel, 2016]. Many of the proinflammatory cytokines implicated in AD pathogenesis use the JAK/STAT pathway for signaling [O'Shea, 2004; Pastore, 2006]. JAK/STAT signaling is utilized by interleukins (IL), interferons, colony-stimulating factors, and growth factors to relay signals from the cell membrane to the nucleus and is indispensable for immune function. JAK3 plays a critical role in T cell development, activation, and proliferation and is predominantly expressed by lymphocytes [Pesu, 2008]. Syk is a member of the family of nonreceptor tyrosine kinases and is involved in regulation of leukocyte immune function, including receptor signaling in mast cells [Choi, 1996], monocytes [Darby, 1994], and T cells [Smith-Garvin, 2009].

Vitiligo is an acquired pigmentary disorder of the skin that is characterized by circumscribed, depiginented macules and patches. The condition is frequently associated with disorders of autoimmune origin, with thyroid abnormalities being the most common. Vitiligo is a condition that causes patchy loss of skin coloring (pigmentation). The average age of onset of vitiligo is in the mid-twenties, but it can appear at any age. It tends to progress over time, with larger areas of the skin losing pigment. Some people with vitiligo also have patches of pigment loss affecting the hair on their scalp or body.

Researchers have identified several forms of vitiligo. Generalized vitiligo (also called nonsegmental vitiligo), which is the most common form, involves loss of pigment (depigmentation) in patches of skin all over the body. Depigmentation typically occurs on the face, neck, and scalp, and around body openings such as the mouth and genitals. Sometimes pigment is lost in mucous membranes, such as the lips. Loss of pigmentation is also frequently seen in areas that tend to experience tubbing, impact, or other trauma, such as the hands, arms, and places where bones are close to the skin surface (bony prominences). Another form called segmental vitiligo is associated with smaller patches of depigmented skin that appear on one side of the body in a limited area; this occurs in about 10 percent of affected individuals.

Vitiligo is generally considered to be an autoimmune disorder. Autoimmune disorders occur when the immune system attacks the body's own tissues and organs. in people with vitiligo the immune system appears to attack the pigment cells (melanocytes) in the skin. About 15 to 25 percent of people with vitiligo are also affected by at least one other autoimmune disorder, particularly autoimmune: thyroid disease, rheumatoid arthritis, type 1 diabetes, psoriasis, pernicious anemia, Addison disease, or systemic lupus erythematosus.

In the absence of other autoimmune conditions, vitiligo does not affect general health or physical functioning. However, concerns about appearance and ethnic identity are significant issues for many affected individuals. Cerdulatinib (DMVT-502, formerly known as RVT-502) is an inhibitor of the JAK family kinases and Syk.

The dual inhibition by DMVT-502 of these two important signaling mechanisms is hypothesized to inhibit the inflammatory process involved in the pathogenesis of AD and may provide relief of signs and symptoms that manifest in the skin. U.S. Pat. Nos. 7,449,456, 8,012,959, 8,138,339, 8,501,944, 8,937,070, and 9,868,729 describe the compound and various methods of treatments thereof. All references cited herein are incorporated in their entirety and for all purposes. Topical application of DMVT-502 is proposed to limit systemic exposure, providing a more favorable safety profile, while targeting delivery to the skin and the underlying inflammation.

### SUMMARY OF THE INVENTION

In a first aspect, the invention provides a topical pharmaceutical formulation comprising:

a) an active agent which treats an inflammatory-related condition, or a pharmaceutically acceptable salt, or a hydrate or a solvate thereof; b) a pharmaceutically acceptable carrier for the active agent; and c) optional preservatives, anti-oxidants, and antimicrobials;

wherein the topical pharmaceutical formulation comprises the active agent, cerdulatinib.

The invention provides additional topical pharmaceutical formulations, as well as methods for their use and production.

In one aspect, the present invention provides a pharmaceutical composition for topical use, comprising cerdulatinib or a pharmaceutically acceptable salt, hydrate or solvate thereof; a pharmaceutically acceptable carrier comprising a polyalkylene glycol having an average molecular weight of from 100 daltons to 10,000 daltons; and propylene glycol. In one aspect, the present invention provides that the active ingredient comprises cerdulatinib as its free base. In one aspect, the present invention provides that the active ingredient comprises cerdulatinib hydrochloride.

In one aspect, the present invention provides a pharmaceutical composition for topical use, comprising cerdulatinib or a pharmaceutically acceptable salt, hydrate or solvate thereof; a pharmaceutically acceptable carrier comprising a polyethylene glycol having an average molecular weight of from 100 daltons to 10,000 daltons; and propylene glycol. In another aspect, the present inventions provides that the polyethylene glycol has an average molecular weight from 100 daltons to 5,000 daltons, or from 200 daltons to 600 daltons. In another aspect, the polyethylene glycol comprises PEG 400.

In one aspect, the present invention provides a pharmaceutical composition for topical use, comprising cerdulatinib or a pharmaceutically acceptable salt, hydrate or solvate thereof; a pharmaceutically acceptable carrier comprising a polyethylene glycol having an average molecular weight from 100 daltons to 10,000 daltons; and propylene glycol and further comprising a penetration enhancer. In another aspect, the penetration enhancer comprises Transcutol HP. In another aspect, the pharmaceutical composition further comprises an antimicrobial preservative and an antioxidant. In another aspect, the antioxidant comprises butylated hydroxytoluene. In another aspect, the antimicrobial preservative comprises phenoxyethanol.

In another aspect, the present invention provides a pharmaceutical composition for topical use, comprising cerdulatinib or a pharmaceutically acceptable salt, hydrate or solvate thereof, a pharmaceutically acceptable carrier comprising a polyethylene glycol having an average molecular weight of from 100 daltons to 10,000 daltons and propylene glycol, wherein the pharmaceutically acceptable carrier further comprises glycerol and/or hydroxypropyl cellulose.

In another aspect, the present invention provides a pharmaceutical composition for topical use, comprising cerdulatinib or a pharmaceutically acceptable salt, hydrate or solvate thereof, a pharmaceutically acceptable carrier comprising a polyethylene glycol having an average molecular weight of from 200 daltons to 600 daltons, propylene glycol, and a penetration enhancer.

In another aspect, the present invention provides a pharmaceutical composition for topical use, comprising cerdulatinib or a pharmaceutically acceptable salt, hydrate or solvate thereof, a pharmaceutically acceptable carrier comprising a polyethylene glycol having an average molecular weight of from 200 daltons to 600 daltons, a polyethylene glycol having an average molecular weight of from 1000 daltons to 10,000 daltons, propylene glycol, and a penetration enhancer. In another aspect, the pharmaceutical composition comprises polyethylene glycol having an average molecular weight from 2,000 daltons to 6,000 daltons. In another aspect, the pharmaceutical composition comprises PEG 4000.

In one aspect, the pharmaceutical composition is a gel. In another aspect, the pharmaceutical composition is an ointment.

In another aspect, the present invention provides a pharmaceutical composition for topical use, comprising:
0.05-1.0% (w/w) of cerdulatinib free base;
30-70% (w/w) of a polyethylene glycol with an average molecular weight of 200 daltons to 600 daltons;
5.0-25% (w/w) of propylene glycol;
5.0-50% (w/w) of a penetration enhancer;
10-35% (w/w) of glycerol and 0.1-3% (w/w) of hydroxypropyl cellulose; and
0.01-1% (w/w) of an antioxidant; and
0.01-2.0% (w/w) of an antimicrobial.

In another aspect, the present invention provides a pharmaceutical composition for topical use, comprising:

0.075-0.75% (w/w) of cerdulatinib free base;

35-60% (w/w) of a polyethylene glycol with an average molecular weight of 200 daltons to 600 daltons;

15-30% (w/w) of a polyethylene glycol with an average molecular weight of 2,000 daltons to 6,000 daltons;

10-20% (w/w) of propylene glycol;

10-20% (w/w) of a penetration enhancer;

0.05-0.25% (w/w) of an antioxidant; and

0.5-1.5% (w/w) of an antimicrobial.

In another aspect, the present invention provides a pharmaceutical composition for topical use, comprising:
0.05-1.0% (w/w) of cerdulatinib hydrochloride;
30-70% (w/w) of a polyethylene glycol with an average molecular weight of 200 daltons to 600 daltons;
5.0-25% (w/w) of propylene glycol;
5.0-50% (w/w) of a penetration enhancer;
10-35% (w/w) of glycerol and 0.1-3% (w/w) of hydroxypropyl cellulose; and
0.01-1% (w/w) of an antioxidant; and
0.01-2.0% (w/w) of an antimicrobial.

In another aspect, the present invention provides a pharmaceutical composition for topical use, comprising:
0.075-0.75% (w/w) of cerdulatinib hydrochloride;
35-60% (w/w) of a polyethylene glycol with an average molecular weight of 200 daltons to 600 daltons;
10-20% (w/w) of propylene glycol;
10-20% (w/w) of a penetration enhancer;
20-30% (w/w) of glycerol and about 1.0% (w/w) of hydroxypropyl cellulose; and
0.05-0.25% (w/w) of an antioxidant; and
0.5-1.5% (w/w) of an antimicrobial.

In another aspect, the present invention provides a pharmaceutical composition for topical use, comprising:
0.075-0.75% (w/w) of cerdulatinib hydrochloride;
40-55% (w/w) of a polyethylene glycol with an average molecular weight of 200 daltons to 600 daltons;
10-20% (w/w) of propylene glycol;
about 15% (w/w) of a penetration enhancer;
20-30% (w/w) of glycerol;
about 1.0% (w/w) of hydroxypropyl cellulose; and
0.05-0.25% (w/w) of an antioxidant; and
0.5-1.5% (w/w) of an antimicrobial.

In another aspect, the present invention provides a pharmaceutical composition for topical use, comprising:
0.075-0.75% (w/w) of cerdulatinib hydrochloride;
35-60% (w/w) of PEG 400;
10-20% (w/w) of propylene glycol;
about 15% (w/w) of a penetration enhancer;
20-30% (w/w) of glycerol;
about 1.0% (w/w) of hydroxypropyl cellulose;
0.05-0.25% (w/w) of an antioxidant; and
0.5-1.5% (w/w) of an antimicrobial.

In another aspect, the present invention provides a pharmaceutical composition for topical use, comprising:
0.05-1.0% (w/w) of cerdulatinib hydrochloride;
30-70% (w/w) of a polyethylene glycol with an average molecular weight of 200 daltons to 600 daltons;
5.0-25% (w/w) of propylene glycol;
5.0-50% (w/w) of a penetration enhancer;
10-35% (w/w) of glycerol;
0.1-3% (w/w) of hydroxypropyl cellulose;
0.01-1% (w/w) of an antioxidant; and
0.01-2.0% (w/w) of an antimicrobial.

In another aspect, the present invention provides a pharmaceutical composition for topical use, comprising:
0.075-0.75% (w/w) of cerdulatinib hydrochloride;
35-60% (w/w) of a polyethylene glycol with an average molecular weight of 200 daltons to 600 daltons;
15-30% (w/w) of a polyethylene glycol with an average molecular weight of 2,000 daltons to 6,000 daltons;
10-20% (w/w) of propylene glycol;
10-20% (w/w) of a penetration enhancer;
0.05-0.25% (w/w) of an antioxidant; and
0.5-1.5% (w/w) of an antimicrobial.

In another aspect, the present invention provides a pharmaceutical composition for topical use, comprising:
0.075-0.75% (w/w) of cerdulatinib hydrochloride;
40-55% (w/w) of a polyethylene glycol with an average molecular weight of 200 daltons to 600 daltons;
20-25% (w/w) of a polyethylene glycol with an average molecular weight of 2,000 daltons to 6,000 daltons;
10-20% (w/w) of propylene glycol;
about 15% (w/w) of a penetration enhancer;
0.05-0.25% (w/w) of an antioxidant; and
0.5-1.5% (w/w) of an antimicrobial.

In another aspect, the present invention provides a pharmaceutical composition for topical use, comprising:
0.075-0.75% (w/w) of cerdulatinib hydrochloride;
35-60% (w/w) of PEG 400;
20-25% (w/w) of PEG 4000;
10-20% (w/w) of propylene glycol;
about 15% (w/w) of a penetration enhancer;
0.05-0.25% (w/w) of an antioxidant; and
0.5-1.5% (w/w) of an antimicrobial.

In another aspect, the present invention provides a pharmaceutical composition for topical use, consisting of:

0.2% (w/w) of cerdulatinib hydrochloride;

44.70% (w/w) of PEG 400;

20.00% (w/w) of propylene glycol;

20.00% (w/w) of glycerol;

13.00% (w/w) of Transcutol HP;

1.00% (w/w) of phenoxyethanol;

1.00% (w/w) of hydroxypropyl cellulose; and

0.10% (w/w) of butylated hydroxytoluene.

In another aspect, the present invention provides a pharmaceutical composition for topical use, consisting of:

0.4% (w/w) of cerdulatinib hydrochloride;

44.50% (w/w) of PEG 400;

20.00% (w/w) of propylene glycol;

20.00% (w/w) of glycerol;

13.00% (w/w) of Transcutol HP;

1.00% (w/w) of phenoxyethanol;

1.00% (w/w) of hydroxypropyl cellulose; and

0.10% (w/w) of butylated hydroxytoluene.

In another aspect, the present invention provides a pharmaceutical composition for topical use, consisting of:

0.1% (w/w) of cerdulatinib hydrochloride;

50.80% (w/w) of PEG 400;

22.00% (w/w) of PEG 4000;

13.00% (w/w) of propylene glycol;

13.00% (w/w) of Transcutol HP;

1.00% (w/w) of phenoxyethanol; and

0.10% (w/w) of butylated hydroxytoluene.

In another aspect, the present invention provides a pharmaceutical composition for topical use, consisting of:

0.2% (w/w) of cerdulatinib hydrochloride;

50.70% (w/w) of PEG 400;

22.00% (w/w) of PEG 4000;

13.00% (w/w) of propylene glycol;

13.00% (w/w) of Transcutol HP;

1.00% (w/w) of phenoxyethanol; and

0.10% (w/w) of butylated hydroxytoluene.

In another aspect, the present invention provides methods for treating a dermatologic condition, comprising topically administering a therapeutically effective amount of a pharmaceutical composition comprising cerdulatinib or a pharmaceutically acceptable salt, hydrate or solvate thereof, to a patient suffering from a dermatologic condition. In one aspect, the dermatologic condition comprises atopic dermatitis. In another aspect, the dermatologic condition comprises moderate to severe atopic dermatitis. In another aspect, the dermatologic condition comprises vitiligo. In one aspect, the flux of cerdulatinib from the composition is greater than 0.2 ng/cm²/hr as determined using a MedFlux-HT™ diffusion cell. In another aspect, the flux of cerdulatinib from the formulation is greater than 0.06 ng/cm²/hr as determined using a MedFlux-HT™ diffusion cell.

### BRIEF DESCRIPTION OF THE DRAWINGS

FIG. 1 is a manufacturing process flow diagram for DMVT-502 HCl salt gels.

FIG. 2 is a manufacturing process flow diagram for DMVT-502 HCl salt ointments.

FIG. 3 is bar chart with the total mean amount (ng) of cerdulatinib recovered from epidermis and dermis following application of 10 formulations.

FIG. 4 is bar chart with the total mean percent applied dose (%) of cerdulatinib recovered from epidermis and dermis following application of 10 formulations.

FIG. 5 is a schematic of the study design used in the DNCB-Induced Atopic Dermatitis in the NC/Nga Mouse Model Study.

FIG. 6a is a bar chart with the total macroscopic score for skin inflammation at day 14 in the NC/Nga mouse model study.

FIG. 6b is a bar chart with the ear thickness at day 14 as change from baseline in the NC/Nga mouse model study.

FIG. 6c is a graph depicting the number of scratches (counts/hr) between day 2 and day 14 in the NC/Nga mouse model study.

FIG. 7 is a graph depicting macroscopic lesion severity scores between day 2 and day 14 in the NC/Nga mouse model study.

FIG. 8 is a bar chart with the serum IgE levels at day 15 in the NC/Nga mouse model study.

FIG. 9 is a collection of bar charts with inflammatory cytokine levels for IL-4, IL-5, IL-13, and IL-31 at day 15 in the NC/Nga mouse model study.

FIG. 10 are plots depicting epidermal thickness and K16 and Ki67 proliferation marker expression.

FIG. 11 are plots depicting infiltration of CD11C+ and CD206+ dendritic cells.

FIG. 12 depicts plots of the effect of Th2 Mediators.

FIG. 13 are plots depicting effects on Th17 Mediators.

FIG. 14 is a plot depicting the correlation of clinical response and immune markers.

FIG. 15 is a diagram of the mouse model of vitiligo used in study DMVT-502-9025.

FIG. 16 is a diagram of the timeline for the vitiligo study.

FIG. 17 is a graph of the vitiligo scores for the vitiligo study.

FIG. 18 is a collection of graphs showing the PMEL cell counts for the vitiligo study.

FIG. 19 is a collection of graphs showing the APC counts for the vitiligo study.

FIG. 20 is a collection of graphs showing the keratinocyte cytokine expression for the vitiligo study.

FIG. 21 is a collection of graphs showing the host T-cell responses for the vitiligo study.

### DETAILED DESCRIPTION

Embodiments of topical formulations for administering a compound are disclosed. Embodiments of methods for preparing the topical formulations are also disclosed. The disclosed formulations are suitable for the treatment of dermatologic conditions such as atopic dermatitis, alopecia areata, vitiligo, and chronic urticaria.

Cerdulatinib hydrochloride, also known as DMVT-502 HCl Salt, is a reversible, small molecule adenosine triphosphate (ATP) competitive inhibitor of the Janus kinase (JAK) family members and nonreceptor spleen tyrosine kinase (Syk) for topical use in the treatment of dermatologic conditions, including for the treatment of patients with moderate to severe atopic dermatitis (AD). Cerdulatnib hydrochloride has the following structure:

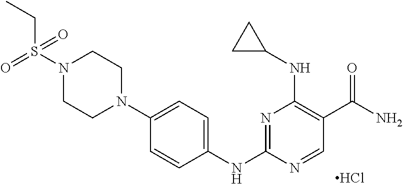 [SMILES: CCS(=O)(=O)N1CCN(c2ccc(Nc3ncc(C(N)=O)c(NC4CC4)n3)cc2)CC1 ; Cl]

### Pharmaceutical Compositions

For topical administration, the composition containing one or more syk and/or JAK inhibitors can be in the form of emulsions, lotions, gels, foams, pastes, creams, jellies, solutions, suspensions, ointments, and transdermal patches. Topically-transdermal patches may also be used. For topical applications, the pharmaceutical compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyethylene glycol, polyoxyethylene, polyoxypropylene compound, emulsifying wax and water. Alternatively, the pharmaceutical compositions may be formulated in a suitable lotion or cream containing the active components suspended or dissolved in one or more pharmaceutically acceptable carriers. Suitable carriers include mineral oil, sorbitan monostearate, polysorbate 60, cetyl esters, wax, cetyl alcohol, 2-octyldodecanol, benzyl alcohol and water. Particular embodiments of the topical formulation comprise a therapeutically effective amount of cerdulatinib or a pharmaceutically acceptable salt, hydrate or solvate thereof and a pharmaceutically acceptable carrier comprising polyethylene glycol and propylene glycol. A person of ordinary skill in the art will appreciate that a therapeutically effective amount of cerdulatinib or a pharmaceutically acceptable salt, hydrate or solvate thereof may vary, but typically the therapeutically effective amount is from 0.01% to 5% (w/w).

The pharmaceutically acceptable carrier may comprise a water-miscible solvent, such as a polyalkylene glycol having an average molecular weight of from 100 daltons to 10,000 daltons. In one aspect, the pharmaceutically acceptable carrier comprises polyethylene glycol having a selected molecular weight. Particular embodiments comprise a polyethylene glycol having an average molecular weight of from 100 to 10,000 daltons as a carrier, preferably 100 to 5,000 Da. According to one aspect, the topical formulation comprises a polyethylene glycol having an average molecular weight of from 200 to 600 daltons, such as PEG 400.

The pharmaceutically acceptable carrier may comprise a mixture of a polyethylene glycol having a molecular weight of from 200 to 600 Da with one or more additional carriers. According to one aspect, the pharmaceutically acceptable carrier further comprises a polyethylene glycol having a molecular weight of from 1,000 to 10,000 daltons, preferably 2,000 to 6,000 Da. In one aspect, the pharmaceutically acceptable carrier comprises PEG 4000. According to another aspect, the pharmaceutically acceptable carrier comprises a polyethylene glycol having a molecular weight of from 200 to 600 Da and propylene glycol. In another aspect, the pharmaceutically acceptable carrier comprises glycerol. In another aspect, the pharmaceutically acceptable carrier comprises hydroxypropyl cellulose.

In an exemplary embodiment, the carrier is an alkylene glycol. In an exemplary embodiment, the pharmaceutically acceptable carrier is propylene glycol, polyethylene glycol, or mixtures thereof. In an exemplary embodiment, the carrier is propylene glycol USP and a polyethylene glycol having a molecular weight of from 200 to 600 Da.

In certain embodiments, the formulation comprises a polyethylene glycol having an average molecule weight from 200 to 600 Da, propylene glycol, and a penetration enhancer and may further comprise a polyethylene glycol having an average molecule weight from 2,000 to 6,000 Da such as PEG4000, glycerol, hydroxypropyl cellulose, an antimicrobial and/or antioxidant.

In certain embodiments, the formulation is an ointment comprising from 0.01% to 3.0% (w/w) cerdulatinib or a pharmaceutically acceptable salt, hydrate or solvate thereof; a pharmaceutically acceptable carrier comprising polyethylene glycol having a molecular weight of from 200 to 600 Da, a polyethylene glycol having a molecular weight of from 2,000 to 6,000 Da, and propylene glycol. In one aspect, the formulation further comprises diethylene glycol monoethyl ether (Transcutol HP).

In certain embodiments, the formulation is a gel formulation comprising from 0.01% to 3.0% (w/w) cerdulatinib or a pharmaceutically acceptable salt, hydrate or solvate thereof; a pharmaceutically acceptable carrier comprising polyethylene glycol having a molecular weight of from 200 to 600 Da, glycerol, and propylene glycol. In one aspect, the formulation further comprises diethylene glycol monoethyl ether (Transcutol HP).

In certain embodiments, the formulation is a gel formulation comprising from 0.01% to 3.0% (w/w) cerdulatinib or a pharmaceutically acceptable salt, hydrate or solvate thereof; a pharmaceutically acceptable carrier comprising polyethylene glycol having a molecular weight of from 200 to 600 Da, and propylene glycol. In one aspect, the formulation further comprises diethylene glycol monoethyl ether (Transcutol HP). In one aspect, the formulation further comprises ethanol. In one aspect, the formulation further comprises benzyl alcohol. In one aspect, the formulation further comprises Tween 80.

The pharmaceutical formulation may also can include antimicrobials such as phenoxythanol; an antioxidant, such as butylated hydroxyanisole, butylated hydroxytoluene, ascorbic acid, a tocopherol, and combinations thereof, with particular embodiments comprising butylated hydroxytoluene as the antioxidant; and a colorant.

For particular embodiments, the therapeutically effective amount is from 0.01% to 5% (w/w), 0.05% to 3% (w/w), 0.05% to 1% (w/w), and 0.075% to 0.75% (w/w) cerdulatinib or a pharmaceutically acceptable salt, hydrate or solvate thereof, and the pharmaceutical formulation further comprises: from 60% to 90% (w/w) of a pharmaceutically acceptable carrier; 10% to 25% of an additional solvent/penetration enhancer; 0.01% to 2.0% of an antimicrobial agent; and from 0.01% to 1.0% (w/w) of an antioxidant.

For particular embodiments the pharmaceutical formulation comprises 0.01% to 5% (w/w), 0.05% to 3% (w/w), 0.05% to 1% (w/w), and 0.075% to 0.75% (w/w) cerdulatinib or a pharmaceutically acceptable salt, hydrate or solvate thereof; a pharmaceutically acceptable carrier comprising from 30% to 70% (w/w) or 35% to 65% (w/w) or 40% to 55% (w/w) polyethylene glycol with an average molecular weight of from 200 to 600 Da; 8% to 25% (w/w) or 10% to 20% (w/w) propylene glycol; and from 5% to 25% (w/w) or 10% to 20% (w/w) of a penetration enhancer. In another aspect, the formulations further comprise 0.01% to 2.0% of an antimicrobial agent; and from 0.01% to 1.0% (w/w) of an antioxidant.

In other disclosed embodiments, the pharmaceutical formulation comprises from 0.05% to 1.0% (w/w) of cerdulatinib or a pharmaceutically acceptable salt, hydrate or solvate thereof; from 40% to 55% (w/w) polyethylene glycol with an average molecular weight of from 200 to 600 daltons; 10% to 20% (w/w) propylene glycol; and 10% to 20% diethylene glycol monoethyl ether (Transcutol HP).

Another embodiment of the pharmaceutical formulation comprises from 0.05% to 1.0% (w/w) of cerdulatinib or a pharmaceutically acceptable salt, hydrate or solvate thereof; from 40% to 55% (w/w) polyethylene glycol with an average molecular weight of from 200 to 600 Da; 10% to 20% (w/w) propylene glycol; 10% to 20% diethylene glycol monoethyl ether (Transcutol HP); 1.0% (w/w) phenoxyethanol; and 0.1% (w/w) butylated hydroxytoluene.

Yet another embodiment of the pharmaceutical formulation comprises from 0.05% to 1.0% (w/w) of cerdulatinib or a pharmaceutically acceptable salt, hydrate or solvate thereof; from 40% to 55% (w/w) polyethylene glycol with an average molecular weight of from 300 to 500 Da; from 15% to 30% (w/w) polyethylene glycol with an average molecular weight of from 2,000 to 6,000 Da; from 10% to 20% (w/w) propylene glycol; from 10% to 20% diethylene glycol monoethyl ether (Transcutol HP); 1.0% (w/w) phenoxyethanol; and 0.1% (w/w) butylated hydroxytoluene.

Yet another embodiment of the pharmaceutical formulation comprises from 0.05% to 1.0% (w/w) of cerdulatinib or a pharmaceutically acceptable salt, hydrate or solvate thereof; from 40% to 55% (w/w) polyethylene glycol with an average molecular weight of from 300 to 500 Da; from 15% to 35% (w/w) glycerol; from 10% to 20% (w/w) propylene glycol; from 10% to 20% diethylene glycol monoethyl ether (Transcutol HP); 1.0% (w/w) phenoxyethanol; and 0.1% (w/w) butylated hydroxytoluene.

Yet another embodiment of the pharmaceutical formulation comprises from 0.05% to 1.0% (w/w) of cerdulatinib or a pharmaceutically acceptable salt, hydrate or solvate thereof; from 40% to 55% (w/w) polyethylene glycol with an average molecular weight of from 300 to 500 Da; from 15% to 35% (w/w) glycerol; from 10% to 20% (w/w) propylene glycol; from 10% to 20% diethylene glycol monoethyl ether (Transcutol HP); from 0.1% to 3% (w/w) hydroxypropyl cellulose; 1.0% (w/w) phenoxyethanol; and 0.1% (w/w) butylated hydroxytoluene.

Yet another embodiment of the pharmaceutical formulation consists of 0.20% (w/w) of cerdulatinib hydrochloride; 44.70% (w/w) polyethylene glycol with an average molecular weight of 400 Da; 20.00% (w/w) glycerol; 20.00% (w/w) propylene glycol; 13.00% diethylene glycol monoethyl ether (Transcutol HP); 1.00% (w/w) hydroxypropyl cellulose; 1.00% (w/w) phenoxyethanol; and 0.10% (w/w) butylated hydroxytoluene.

Yet another embodiment of the pharmaceutical formulation consists of 0.40% (w/w) of cerdulatinib hydrochloride; 44.50% (w/w) polyethylene glycol with an average molecular weight of 400 Da; 20.00% (w/w) glycerol; 20.00% (w/w) propylene glycol; 13.00% diethylene glycol monoethyl ether (Transcutol HP); 1.00% (w/w) hydroxypropyl cellulose; 1.00% (w/w) phenoxyethanol; and 0.10% (w/w) butylated hydroxytoluene.

Yet another embodiment of the pharmaceutical formulation consists of 0.10% (w/w) of cerdulatinib hydrochloride; 50.80% (w/w) polyethylene glycol with an average molecular weight of 400 Da; 22.00% (w/w) polyethylene glycol with an average molecular weight of 4,000 Da; 13.00% (w/w) propylene glycol; 13.00% diethylene glycol monoethyl ether (Transcutol HP); 1.00% (w/w) phenoxyethanol; and 0.10% (w/w) butylated hydroxytoluene.

Yet another embodiment of the pharmaceutical formulation consists of 0.20% (w/w) of cerdulatinib hydrochloride; 50.70% (w/w) polyethylene glycol with an average molecular weight of 400 Da; 22.00% (w/w) polyethylene glycol with an average molecular weight of 4,000 Da; 13.00% (w/w) propylene glycol; 13.00% diethylene glycol monoethyl ether (Transcutol HP); 1.00% (w/w) phenoxyethanol; and 0.10% (w/w) butylated hydroxytoluene.

A person of ordinary skill in the art will appreciate that the pharmaceutical formulation may also comprise a therapeutically effective amount of an additional or subsequent active agent, or agents.

A person of ordinary skill in the art also will appreciate that the pharmaceutical formulation may comprise other agents, such as a fragrance, an absorbent, an astringent, a binder, a buffering agent, a chelating agent, a film-forming agent, a conditioning agent, an opacifying agent, a protectant, or any combination thereof.

Certain embodiments concern a method for treating a dermatological disorder. For example, the method may comprise topically administering to a subject disclosed embodiments of the pharmaceutical formulation. Dermatological disorders include atopic dermatitis, alopecia areata, vitiligo, and chronic urticaria. For particular embodiments, the method comprises identifying a subject having atopic dermatitis. A disclosed embodiment, or embodiments, of the pharmaceutical formulation is applied topically. The disclosed method contemplates using any one of the disclosed embodiments of the pharmaceutical formulation in treating dermatological disorders.

The DMVT-502 topical gel or ointment preparations are applied locally at the area of the lesion. Preferably, the preparations are applied locally after bathing and toweling dry, and the subject does not bathe for at least 2 hours after application of topical DMVT-502 preparations. Topical application of the formulations may be applied one or more times daily, such as twice daily. The topical DMVT-502 preparations are not suitable for applying around the eyes.

The foregoing and other objects, features, and advantages of the invention will become more apparent from the following detailed description.

In an exemplary embodiment, the topical pharmaceutical formulation further comprises an antioxidant. In an exemplary embodiment, the antioxidant is selected from the group consisting of butylated hydroxytoluene, ascorbic acid, ascorbic palmitate, butylated hydroxyanisole, 2,4,5-trihydroxybutyrophenone, 4-hydroxymethyl-2,6-di-fe/f-butylphenol, erythorbic acid, gum guaiac, propyl gallate, thiodipropionic acid, dilauryl thiodipropionate, tert-butylhydroquinone and a tocopherol, or a pharmaceutically acceptable salt or ester thereof, or a combination thereof. In an exemplary embodiment, the antioxidant is butylated hydroxytoluene. In an exemplary embodiment, the antioxidant is butylated hydroxytoluene NF.

In an exemplary embodiment, the antioxidant is present in a concentration of about 0.01% (w/w) to about 1.5% (w/w). In an exemplary embodiment, the antioxidant is present in a concentration of about 0.10% (w/w) to about 1.0% (w/w). In an exemplary embodiment, the antioxidant is present in a concentration of about 1.0% (w/w). In an exemplary embodiment, the antioxidant is present in a concentration of 1.0% (w/w).

According to one aspect, a gel formulation of cerdulatinib hydrochloride, described as a colorless to yellow, clear stringy gel of medium viscosity and smooth application, is provided. According to another aspect, an ointment formulation of cerdulatinib hydrochloride is provided, described as an opaque, white to yellow ointment of high viscosity and smooth application.

The gel drug product formulation has an DMVT-502 HCl Salt bulk drug content of 0.1% (0.09% free base), 0.2% (0.18% free base), or 0.4% (0.37% free base). The ointment drug product formulation has an DMVT-502 HCl Salt bulk drug content of either 0.1% or 0.2%.

### Manufacturing Process

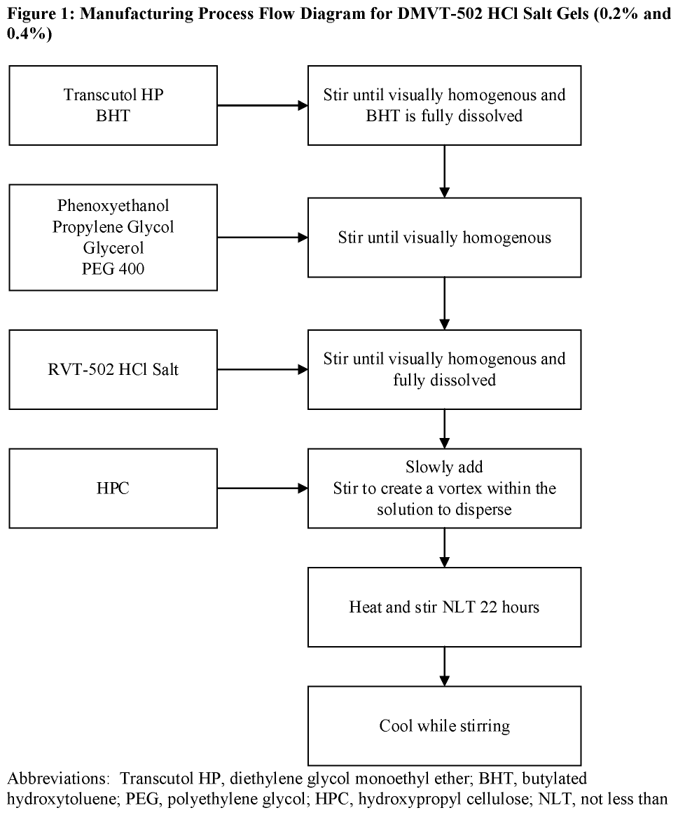

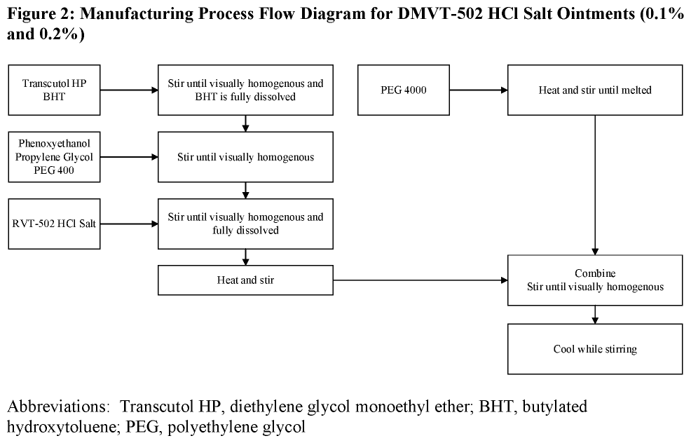

The gel and ointment drug products are manufacturing using conventional blending, melting, mixing and cooling processes. The flow diagrams for the process for preparing gels and ointments according to the invention are depicted in FIGS. 1 and 2.

The following examples are given by way of illustration and not by way of limitation.

### EXAMPLE 1

### In Vitro Potency and Selectivity of DMVT-502

In vitro pharmacology studies have evaluated the activity and potency of DMVT-502 against a panel of purified kinase assays followed by specific cellular potency assays against Syk, JAK1, JAK2, JAK3, and tyrosine kinase 2 (Tyk2). The potency of DMVT-502 was assessed in primary cells and whole blood stimulated with a variety of cytokines to measure JAK/signal transducer and activator of transcription (STAT)-specific pathway responses.

Potency against Syk, JAK1, JAK2, JAK3, and Tyk2 were tested using the DiscoveRx cellular platform and compared to tofacitinib and the JAK1/2 selective inhibitor, ruloxitinib. Table 1 indicates significant potency against Syk, JAK1, and Tyk2, with diminished potency against cellular JAK2 and JAK3.

TABLE 1
Potency of DMVT-502 against Syk, JAK1, JAK2, JAK3, and
Tyk2 in a DiscoveRx Cellular Platform

| | DiscoveRx Cellular IC₅₀ (μM) | | | | |
|---|---|---|---|---|---|
| | Syk | JAK1 | JAK2 | JAK3 | Tyk2 |
| DMVT-502 | 1.28 | 0.098 | 4.86 | 5.14 | 1.72 |
| CP690,550^{a} | — | — | — | 1.23 | — |
| INCB018424^{b} | — | 0.054 | 0.35 | — | 1.98 |

Abbreviation: IC₅₀, 50% inhibitory concentration
^{a}Tofacitinib
^{b}Ruloxitinib

### EXAMPLE 2

### Inhibition of Cytokine Signaling in Peripheral Blood Mononuclear Cell Cultures and Human Whole Blood

Human primary cells isolated from healthy volunteers were stimulated with a variety of cytokines to measure JAK/STAT dependent or independent signaling and functional responses following exposure to DMVT-502. Peripheral blood mononuclear cells were prepared from human whole blood and incubated with various concentrations of DMVT-502 prior stimulation with the appropriate cytokine to initiate JAK/STAT signaling. Cells were fixed, permeabilized, and subsequently stained with cell specific lineage and phosphorylated-STAT antibodies for intracellular phospho-flow cytometry to determine the effect of DMVT-502 on cytokine-mediated STAT phosphorylation. Data from these assays confirm that DMVT-502 is a potent inhibitor of JAK1/JAK3-dependent signaling pathways with IC₅₀ values of less than 0.2 μM in T cells and monocytes.

Cytokine stimulations were also performed in human whole blood to estimate the potency of DMVT-502 against JAK/STAT signaling following dosing in humans. To evaluate downstream signaling following cytokine stimulation, human whole blood was stimulated with IL-2 (JAK1/3-mediated signaling), which results in phosphorylation of STAT5 at tyrosine residue 694 (Y694). Inhibition of JAK/STAT signaling following exposure to DMVT-502 was measured in T cells via phospho-flow cytometry. The DMVT-502 IC₅₀ values were 0.3 μM and 0.16 μM in CD4+ and CD8+ T cells, respectively. IL-4 (JAK1/3-mediated) stimulation results in phosphorylation of STAT6 Y641 in CD4+ T cells, CD8+ T cells, CD14+ monocytes, and CD19+ B cells; DMVT-502 inhibited IL-4 mediated signaling with IC₅₀ values of 0.58 μM, 0.33 μM, 0.998 μM, and 0.92 μM, respectively in these various cell types. IL-6 (JAK1/2/Tyk2) stimulation leads to STAT3 Y705 phosphorylation in monocytes. STAT3 Y705 phosphorylation was inhibited by DMVT-502 with an IC₅₀ of 0.26 μM, whereas granulocyte-macrophage colony-stimulating factor (GM-CSF) stimulation (JAK2-mediated) induced STAT5 Y694 phosphorylation in monocytes was not potently inhibited by DMVT-502 (˜4 μM), again indicating the enhanced potency against JAK1/3 and Tyk2-dependent signaling pathways relative to JAK2-mediated cellular signaling.

Dendritic cells (DCs) are important antigen presenting cells that play an integral role in mediating inflammatory skin diseases. A subset of DCs are derived from monocytes and this differentiation is driven by IL-4/GM-CSF co-stimulation. The ability of DMVT-502 to disrupt the signaling responsible for monocyte differentiation into immature DCs was assessed by flow cytometry. Purified monocytes were subsequently cultured with DMVT-502 at various concentrations prior to IL-4/GM-CSF co-stimulation. Five days later, cells were stained for CD14 (monocyte marker) and CD1a (immature dendritic cell marker) and assessed for immature DC differentiation. DMVT-502 inhibited IL-4/GM-CSF-mediated monocyte differentiation to immature DCs with an IC₅₀ of ˜0.1 μM, as evidenced by the decreased expression of CD1a with increasing DMVT-502 concentration. These data suggest that DMVT-502 has the potential to affect antigen presentation in vivo. Similarly, following IL-4 stimulation several cell surface activation markers are upregulated in leukocytes. DMVT-502 was also found to inhibit the IL-4-mediated upregulation of the cell surface markers CD23 (low affinity immunoglobulin [Ig]E receptor) and CD25 (IL-2 receptor alpha chain), as assessed by flow cytometry, on monocytes with IC₅₀ values of 0.23 and 0.42 μM, respectively.

### EXAMPLE 3

### Skin Permeation Study

A study was conducted is to assess the in vitro skin permeation and penetration of cerdulatinib. The study included full scale in vitro skin permeation and penetration experiments for formulations containing cerdulatinib.

Freshly excised human skin (dermatomed to 500±50 μm thickness) from one skin donor was mounted between the donor and receptor compartment of the MedFlux-HT™ diffusion cell (with an exposed dosing surface area of ˜1 cm² for each replicate). The skin was dosed with ca. 10 mg with the cerdulatinib formulations to achieve a dose of ˜10 mg/cm². The pump of the MedFlux system was adjusted to maintain a continuous receiver fluid flow-rate of approximately 10 μL/min (600 μL/hr) directly under the skin. Receiver fluid was automatically collected into a 96-well plate at 2 hour intervals over the course of 24 h and analysed using an LC-MS/MS analytical method.

Following the 24 h in vitro drug permeation experiment, the residual formulation was removed from the surface of the skin and then the skin surface was taped striped up to 5 times to remove the Stratum corneum. The epidermis was then heat-separated from the dermis by placing the skin into an incubator at 60° C. for 2 min, followed by manually separation using gloved hands.

The compositions of the 10 formulations tested are shown below in Table 2.

TABLE 2
Composition of the 10 formulations tested in the skin permeation study

| | Theoretical composition of pharmaceutically acceptable carriers Active (% w/w) | | | | | | | | | | |
|---|---|---|---|---|---|---|---|---|---|---|---|
| Excipient | AG78 | NA24 | NA55 | NA56 | NA57 | NA65 | NA79 | NA80 | NA82 | PO3 | PO4 |
| Cerdulatinib | 0.18 | 0.25 | 0.21 | 0.14 | 0.28 | 0.4 | 0.192 | 0.4 | 0.3 | 0.2 | 0.24 |
| Propylene glycol | 15 | 20 | 10 | 20 | 15 | 20 | — | 20 | 20 | 13 | 16 |
| Transcutol HP | 8.5 | 20 | 20 | 20 | 15 | 13 | 21 | 8 | — | 13 | 6.4 |
| Tween 80 | — | 5 | — | — | — | — | — | — | — | — | — |
| PEG 400 | 64.22 | 41.65 | 57.69 | 57.76 | 57.62 | 44.5 | 66.708 | 39.6 | 68 | 50.7 | 51.26 |
| Ethanol | — | 10 | 10 | — | 10 | — | 10 | — | 10 | — | — |
| Glycerol | | — | — | — | — | 20 | — | 30 | — | — | — |
| Benzyl alcohol | — | 2 | — | — | — | — | — | — | — | — | — |
| Phenoxyethanol | 1 | — | 1 | 1 | 1 | 1 | 1 | 1 | 0.7 | 1 | 1 |
| BHT | 0.1 | 0.1 | 0.1 | 0.1 | 0.1 | 0.1 | 0.1 | 0.1 | — | 0.1 | 0.1 |
| PEG 4000 | — | — | — | — | — | — | — | — | — | 22 | 25 |
| HPC-HF Pharm | 1 | 1 | 1 | 1 | 1 | 1 | 1 | 1 | 1 | — | — |
| Total | 100 | 100 | 100 | 100 | 100 | 100 | 100 | 100.1 | 100 | 100 | 100 |

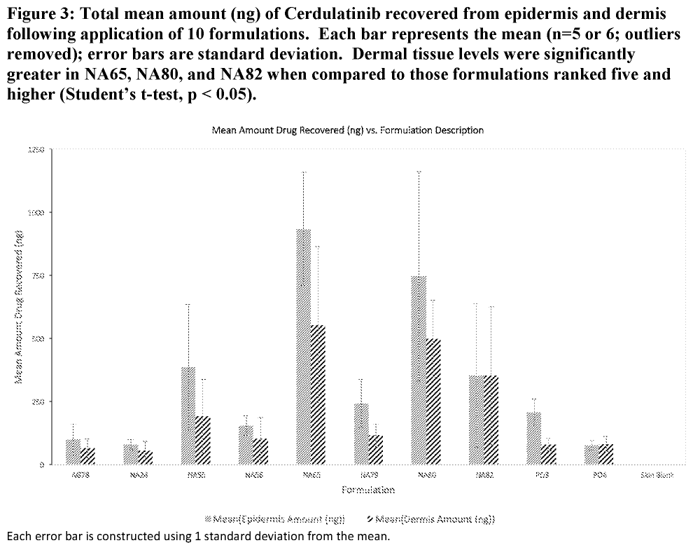

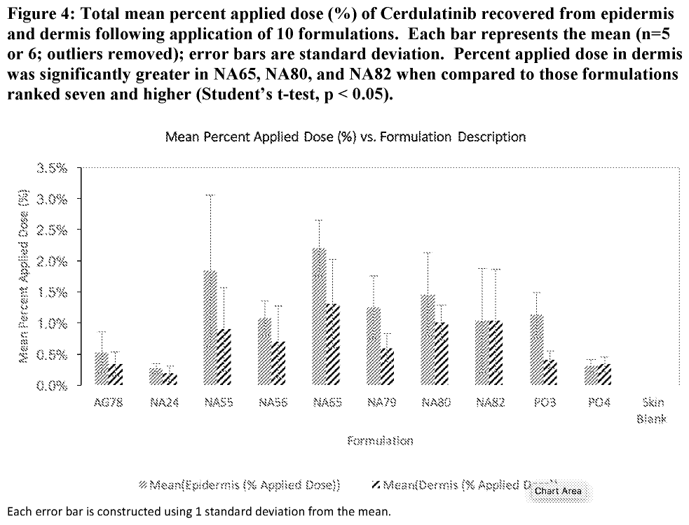

The results of the skin permeation study are presented in Tables 3 and 4 below and FIGS. 3 and 4.

TABLE 3
Mean cumulative amount (ng/cm²) of Cerdulatinib permeated
through the 1 cm² skin dosing area (i.e. drug detected in the
PBS + 0.01% Tween 20 receiver fluid) following application of
10 formulations of Cerdulatinib. NA65 was significantly
greater than rank 4 and higher; NA80 and PO4 were significantly
greater than rank 10 (Student's t-test).

| Formulation | % API (w/w) | % Applied Dose | Flux (ng/cm²/hr) |
|---|---|---|---|
| AG78 | 0.18 | 0.00364% | 0.0210 |
| NA24 | 0.25 | 0.00041% | 0.0120 |
| NA55 | 0.21 | 0.00132% | 0.0311 |
| NA56 | 0.14 | 0.00168% | 0.0139 |
| NA65 | 0.4 | 0.01238% | 0.2127 |
| NA79 | 0.192 | 0.00237% | 0.0179 |
| NA80 | 0.4 | 0.00311% | 0.1328 |
| NA82 | 0.3 | 0.00000% | 0.0000 |
| PO3 | 0.2 | 0.00516% | 0.0611 |
| PO4 | 0.24 | 0.00728% | 0.1230 |

TABLE 4
Full scale data penetration results

| | | | | | | | | | | | | | Epidermis | | | Dermis | | |
|---|---|---|---|---|---|---|---|---|---|---|---|---|---|---|---|---|---|---|
| | Epidermis | | | Dermis | | | Epidermis (% | | | Dermis (% | | | Concentration | | | Concentration | | |
| | Amount (ng) | | | Amount (ng) | | | Applied Dose) | | | Applied Dose) | | | (ug/g)* | | | (ug/g)* | | |
| | | | Std | | | Std | | | Std | | | Std | | | Std | | | Std |
| Formulation | N | Mean | Dev | N | Mean | Dev | N | Mean | Dev | N | Mean | Dev | N | Mean | Dev | N | Mean | Dev |
| AG78 (USTR-2017- | 6 | 100.2 | 61.0 | 5 | 64.8 | 37.7 | 6 | 0.53% | 0.33% | 5 | 0.34% | 0.19% | 6 | 11.79 | 7.17 | 5 | 0.675 | 0.392 |
| 0250) | | | | | | | | | | | | | | | | | | |
| NA24 (USTR-2017- | 5 | 80.4 | 20.2 | 6 | 56.1 | 36.4 | 5 | 0.27% | 0.08% | 6 | 0.19% | 0.12% | 5 | 9.46 | 2.38 | 6 | 0.584 | 0.379 |
| 0203) | | | | | | | | | | | | | | | | | | |
| NA55 (USTR-2017- | 6 | 387.0 | 249.4 | 6 | 193.2 | 145.1 | 6 | 1.84% | 1.22% | 6 | 0.90% | 0.67% | 6 | 45.53 | 29.34 | 6 | 2.012 | 1.511 |
| 0195) | | | | | | | | | | | | | | | | | | |
| NA56 (USTR-2017- | 6 | 155.3 | 39.2 | 6 | 102.2 | 85.4 | 6 | 1.08% | 0.28% | 6 | 0.70% | 0.58% | 6 | 18.27 | 4.61 | 6 | 1.065 | 0.889 |
| 0205) | | | | | | | | | | | | | | | | | | |
| NA65 (USTR-2017- | 6 | 934.3 | 224.7 | 6 | 555.0 | 308.0 | 6 | 2.20% | 0.45% | 6 | 1.31% | 0.71% | 6 | 109.92 | 26.43 | 6 | 5.782 | 3.208 |
| 0207) | | | | | | | | | | | | | | | | | | |
| NA79 (USTR-2017- | 6 | 241.9 | 96.0 | 5 | 116.2 | 45.0 | 6 | 1.25% | 0.50% | 5 | 0.60% | 0.23% | 6 | 28.46 | 11.29 | 5 | 1.210 | 0.469 |
| 0209) | | | | | | | | | | | | | | | | | | |
| NA80 (USTR-2017- | 6 | 746.2 | 415.6 | 5 | 499.6 | 151.6 | 6 | 1.46% | 0.67% | 5 | 1.01% | 0.28% | 6 | 87.78 | 48.90 | 5 | 5.204 | 1.579 |
| 0254) | | | | | | | | | | | | | | | | | | |
| NA82 (USTR-2017- | 6 | 353.7 | 284.3 | 6 | 352.5 | 273.5 | 6 | 1.04% | 0.84% | 6 | 1.04% | 0.83% | 6 | 41.62 | 33.45 | 6 | 3.672 | 2.849 |
| 0252) | | | | | | | | | | | | | | | | | | |
| PO3 (USTR-2017- | 5 | 207.9 | 52.3 | 6 | 79.1 | 25.2 | 5 | 1.13% | 0.36% | 6 | 0.41% | 0.14% | 5 | 24.45 | 6.15 | 6 | 0.823 | 0.263 |
| 0211) | | | | | | | | | | | | | | | | | | |
| PO4 (USTR-2017- | 6 | 77.6 | 20.6 | 6 | 82.8 | 29.6 | 6 | 0.32% | 0.10% | 6 | 0.34% | 0.12% | 6 | 9.13 | 2.43 | 6 | 0.863 | 0.308 |
| 0248) | | | | | | | | | | | | | | | | | | |
| Skin Blank | 1 | 0.0 | — | 1 | 0.0 | — | 0 | — | — | 0 | — | — | 1 | 0.00 | — | 1 | 0.000 | — |

The study showed that Cerdulatinib was generally detectable at low levels in receiver fluid (method LLOQ of 0.0500 ng/mL). Most formulations were not significantly different from one another in receiver fluid. NA65 had significantly greater flux with respect to formulations ranked 4 and higher. NA80 and PO4 had significantly greater flux with respect only to NA82. On an amount (ng) basis, non-aqueous gel formulations NA65, NA80, and NA82 delivered significantly greater amounts of API to the dermis (555, 500, 352 ng, respectively) with respect to formulations ranked five and higher. On a percent applied dose basis, non-aqueous gel formulations NA65, NA80, and NA82 delivered significantly greater amounts of API to the dermis (1.3%, 1.0%, and 1.0%, respectively) with respect to formulations ranked seven and higher.

### EXAMPLE 4

### Stability

Stability results to date from lab scale development batches demonstrate that DMVT-502 HCl Salt gel (0.4%) and ointment (0.2%) is stable for up to 3 months at 25° C./60% RH and at 40° C./75% RH. Throughout the 3-month time period, lab scale development batch stability results met the shelf life specifications established for the gel and ointment clinical batches. There was no change in appearance (microscopic and macroscopic) on stability. Assay results did not show a decreasing trend over time. The intended long-term storage condition for the gel and ointment drug products is room temperature. Stability for gel formulations has further been shown to remain within specifications at 12 months.

### EXAMPLE 5

### DNCB-Induced Atopic Dermatitis in the NC/Nga Mouse Model

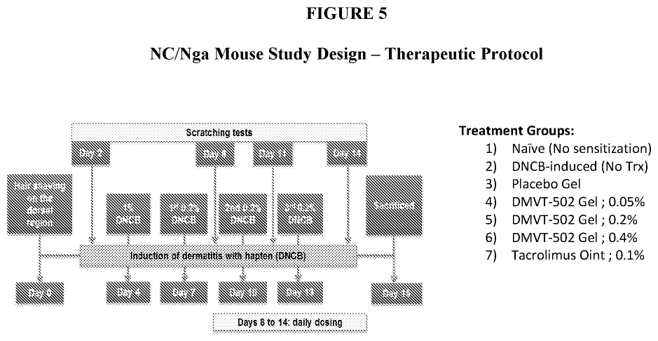

Gross skin inflammation, ear thickness, and scratching behavior of mice treated with the NA65 cerdulatinib hydrochloride gel formulation, placebo, and a tacrolimus ointment were evaluated in the NC/Nga mouse model (Suto H. et al. NC/Nga Mice: A Mouse Model for Atopic Dermatitis, Int Arch Allergy Immunol 1999; 120 (suppl 1): 70-75). The NA65 gel formulation of Table 2, Example 3, was formulated at 0.05%, 0.2%, and 0.4% cerdulatinib hydrochloride and tested in the NC/Nga mouse model along with a placebo gel and a 0.1% tacrolimus ointment according to the protocol of FIG. 5. Atopic dermatitis was induced by repeated application of DNCB (1-chloro-2,4-dinitrobenzene) to the dorsal skin of the ears/back. DNCB sensitization resulted in atopic dermatitis-like symptoms as observed historically. Mice were treated with 0.05%, 0.2%, or 0.4% cerdulatinib formulations daily on days 8-14. Lesions were evaluated on days 2, 8, 11, and 14.

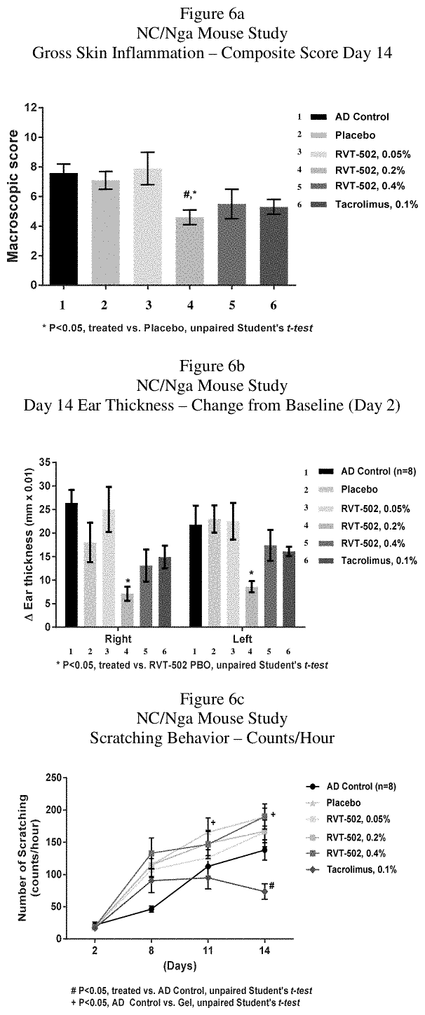

Composite scores for gross skin inflammation at day 14 are depicted in FIG. 6a, ear thickness—change from baseline are shown in FIG. 6b, and scratching behavior (counts/hr) is shown in FIG. 6c. Skin lesion scores were significantly improved with the two highest concentrations of cerdulatinib on Day 14 relative to atopic dermatitis controls. Treatment with Placebo Gel demonstrated a modest effect on gross inflammation parameters. 0.2% cerdulatinib gel treatment resulted in a statistically significant reduction in gross inflammation vs. atopic dermatitis control animals at all timepoints. In general, treatment with 0.2% cerdulatinib gel resulted in a greater inhibition of inflammation.

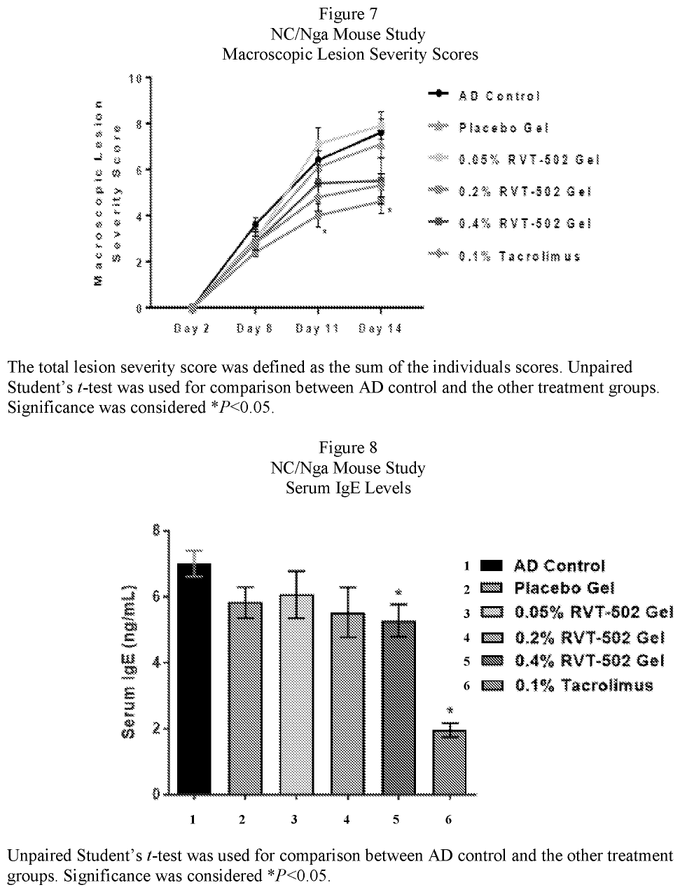

Macroscopic lesion severity scores are depicted in FIG. 7. Macroscopic skin lesion severity was measured on the indicated Study Days by assessing (0-3 scale) the presence of erythema, edema, excoriation/erosion, and dryness scaling on the ears, neck, and dorsal skin of animals.

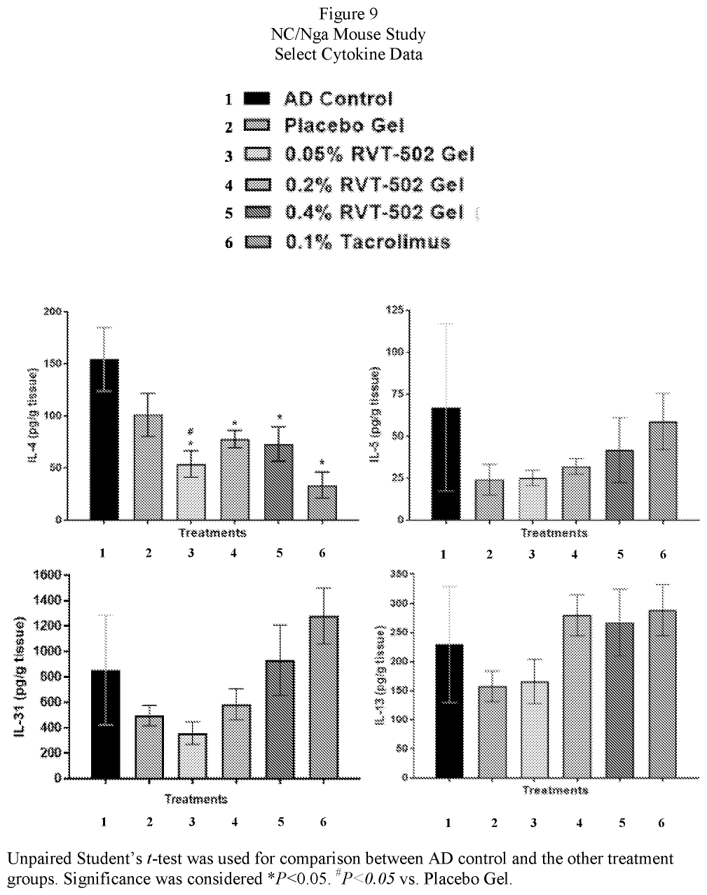

Serum IgE levels are depicted in FIG. 8. Serum samples obtained on Study Day 15 were utilized for IgE quantitation. Select cytokine data from the study are depicted in FIG. 9. Skin samples harvested on Day 15 were analyzed by LUMINEX for inflammatory cytokine levels for IL-4, IL-5, IL-13, and IL-31.

In summary, significant reductions in ear thickness and macroscopic lesion severity scores were observed with 0.2% DMVT-502 Gel on Study Days 11 and 14 in this therapeutic study design. In addition, the study showed trends towards reduction in serum IgE levels following topical DMVT-502 therapy. This therapeutic model also revealed limited suppression of pro-inflammatory cytokine levels following topical treatment with either tacrolimus or DMVT-502 therapy. These results indicate that DMVT-502 may be an effective therapy for AD.

### EXAMPLE 6

### DMVT-502-1001 First in Human Topical Administration

DMVT-502-1001 is an ongoing Phase 1 (clinical phase complete), first-in-human (for topical administration of DMVT-502) study that enrolled healthy adult subjects and subjects with atopic dermatitis in Canada. The study is evaluating the safety, tolerability, and pharmacokinetics of DMVT-502 topical formulations (gel and ointment) after single and multiple dosing.

A total of 42 subjects were enrolled and exposed to at least one dose of study medication (active or vehicle). Of these, 40 subjects were exposed to at least one application of active DMVT-502 ointment or gel (32 healthy subjects and 8 subjects with AD) and 2 subjects with AD received vehicle. DMVT-502 gel was applied to a body surface area (BSA) of 8% twice daily for 10 days in healthy subjects and was applied up to 10% BSA twice daily for 14 days in AD subjects. Eight of the 10 subjects with AD were exposed to 0.37% DMVT-502 gel (measured as free base DMVT-502), administered BID for 14 days.

Systemic exposure following topical administration of 0.18% and 0.37% DMVT-502 topical gel in healthy subjects from the Phase 1 study were all below level of quantification (<250 pg/mL) after twice daily dosing over 8% BSA, and adverse events were mild to moderate in severity and all were listed as resolving or resolved by end of treatment.

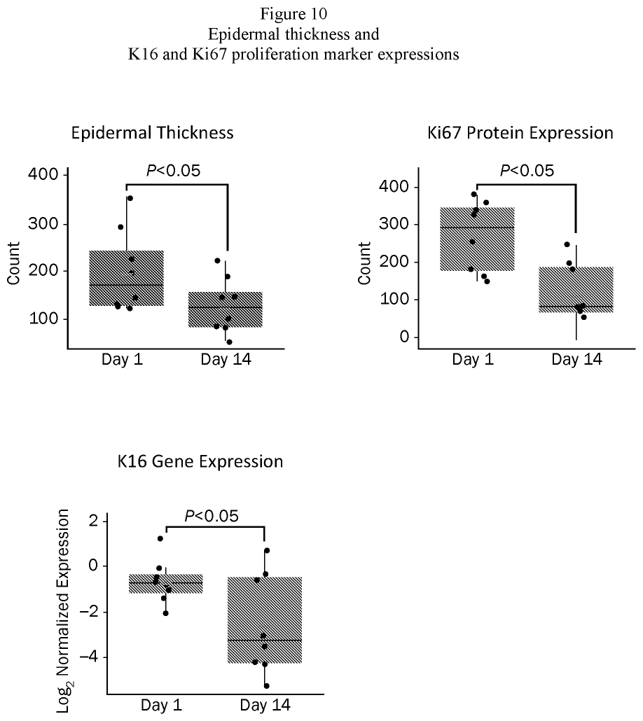

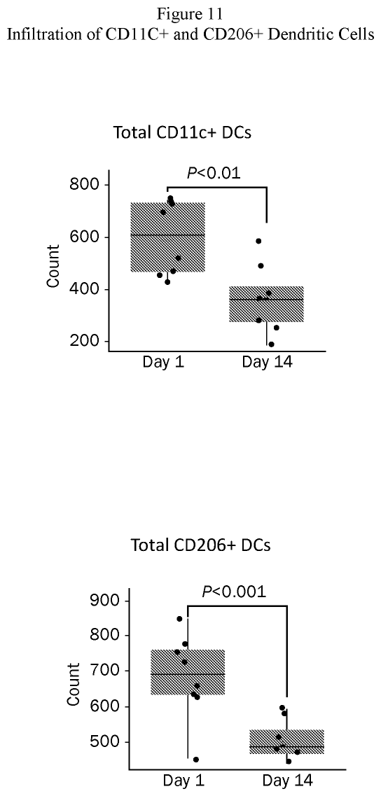

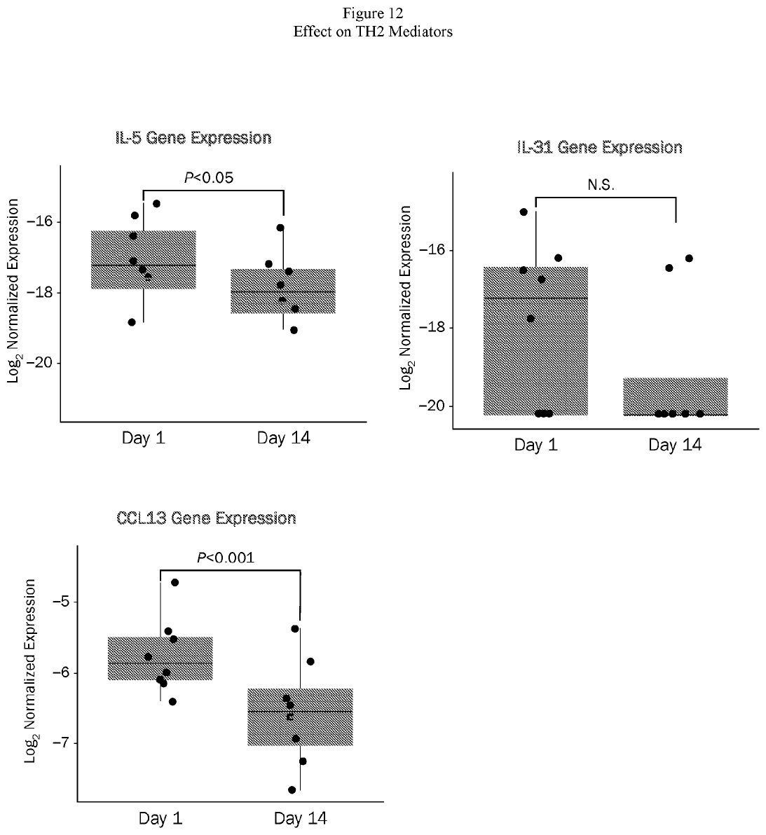

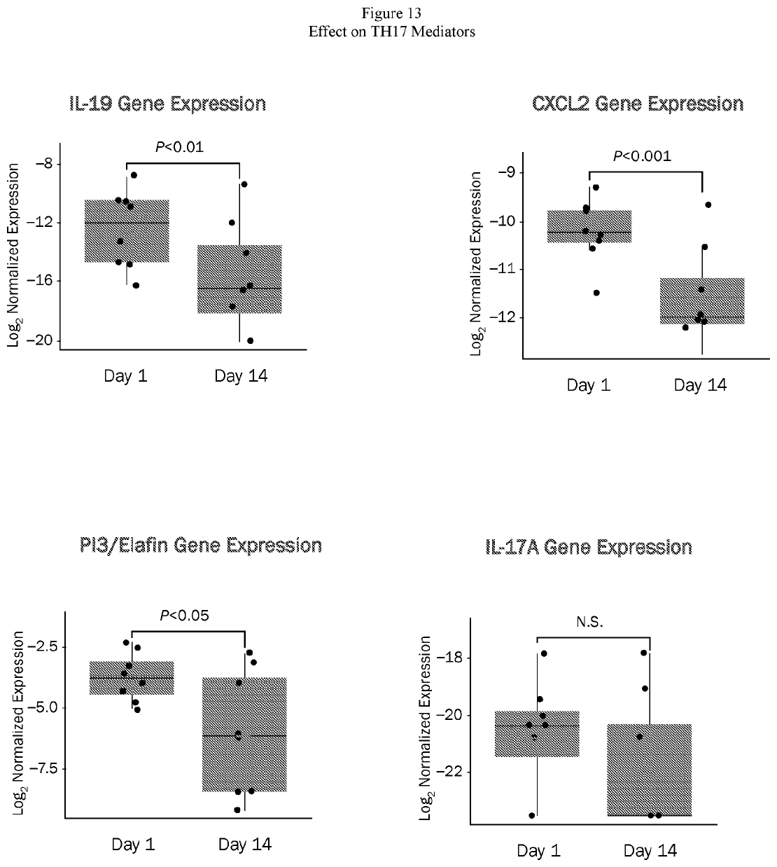

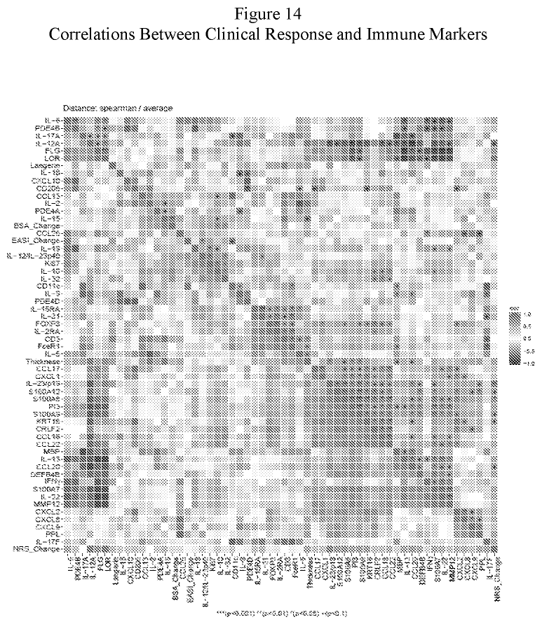

Patients (n=8) with AD (median age 28.5 years old) were treated with topical cerdulatinib. Baseline mean Eczema Area Severity Index (EASI) score was 4.0, mean BSA score was 4.3% and mean pruritis NRS value was 4.4. After 14 days of treatment, EASI, BSA and NRS scores improved significantly from baseline, by 65% (P<0.001), 54% (P=0.022), and 64%, respectively. There were significant improvements (P<0.05) in measures of epidermal hyperplasia, including epidermal thickness and K16 and Ki67 proliferation marker expression, compared with baseline (all P<0.05) as depicted in FIG. 10. As shown in FIG. 11, infiltration of CD11c+ dendritic cells (DC) and CD206+ inflammatory epidermal DCs were significantly reduced compared with baseline (both P<0.01), with similar trends observed for FccRI+ DCs and CD3+ T cells. Reduced cellular infiltrates were associated with significant reductions from baseline in gene expression of immune markers including: inflammatory marker matrix metalloproteinase 12; innate mediators interleukin (IL)-6 (P<0.001) and IL-8 (P<0.01); Th2 mediators IL-5 (P<0.05), IL-31 and CCL13 (P<0.001) (See FIG. 12); Th17 mediators IL-17, IL-19 (P<0.01), CXCL2 (P<0.001) and Elafin/PI3 (P<0.05) (See FIGS. 13); and Th17/Th22-associated genes S100A7, S100A8, SA100A9, S100Al2 (all P<0.05). As seen in Tables 5-7 below and in FIG. 14, changes in the levels of cellular and molecular immune markers were also associated with improvement in clinical response.

TABLE 5
EASI Correlation

| | EASI Correlaton | EASI P-value |
|---|---|---|
| IL23p40 | 0.738095 | 0.045833 |
| Ki67 | 0.547619 | 0.710982 |
| IL32 | 0.500000 | 0.216171 |
| CCL13 | 0.428571 | 0.299206 |
| CD206 | 0.428571 | 0.299206 |

TABLE 6
BSA Correlation

| | BSA Correlation | BSA P-value |
|---|---|---|
| IL15 | 0.902708 | 0.002138 |
| IL10 | 0.756323 | 0.029884 |
| IL32 | 0.731925 | 0.038998 |
| PDE4A | 0.609938 | 0.108350 |
| IL2 | 0.585540 | 0.127247 |
| Ki67 | 0.561143 | 0.147860 |
| IL23p40 | 0.561143 | 0.147860 |
| CCL13 | 0.487950 | 0.219944 |
| CRLF2 | 0.463553 | 0.247323 |
| CCL18 | 0.414758 | 0.306912 |

Pruritis Correlation

| | Pruritus NRS | BSA |
|---|---|---|
| | Correlation | P-value |
| IL23p19 | 0.834749 | 0.009930 |
| S100A8 | 0.711991 | 0.047567 |
| KRT16 | 0.662889 | 0.073194 |
| CCL20 | 0.650613 | 0.080638 |
| S100A9 | 0.638337 | 0.088505 |
| S100A12 | 0.638337 | 0.088505 |
| IL17F | 0.592773 | 0.121465 |
| PPL | 0.589234 | 0.124275 |
| S100A7 | 0.589234 | 0.124275 |
| PI3 | 0.589234 | 0.124275 |
| Thickness | 0.589234 | 0.124275 |
| DEFB4B | 0.576959 | 0.134301 |
| IL13 | 0.576959 | 0.134301 |
| CXCL2 | 0.527856 | 0.178749 |
| CXCL8 | 0.515580 | 0.190942 |

All treatment-related adverse events were Grade 1 (34/35 events; 97%) or Grade 2 (1/35 events; 3%) and most were resolved by the end of the study with no safety related withdrawals. Topical cerdulatinib BID for 14 days was well tolerated in patients with AD. Significant clinical improvements of AD in response to topical cerdulatinib were associated with tissue reversal of epidermal hyperplasia, reduced immune-cell infiltration and AD-related inflammatory gene expression.

### EXAMPLE 7

### A Phase 2 Randomized, Double-Blind, Vehicle-Controlled, Dose-Ranging Study to Evaluate the Efficacy, Safety, and Tolerability of Topical DMVT-502 (cerdulatinib) Gel in Adult and Adolescent Subjects with Atopic Dermatitis

This is a Phase 2 randomized, double-blind, vehicle-controlled, dose-ranging study to evaluate the efficacy, safety, and tolerability of topical DMVT-502 gel in adults and adolescents with atopic dermatitis. Study duration for completed subjects is approximately 17 weeks in total. Twice daily applications should be at least 8 hours apart. Study treatment should be applied to dry, clean skin.

There are 4 periods to the study:

### 1. Screening

### 2. Baseline

### 3. Double-Blind Treatment

a. Vehicle-Control Treatment Phase
b. Optional Active Treatment Phase

### 4. Follow-Up (or Early Termination)

During the Vehicle-Control Treatment Phase the subjects will receive either topical DMVT-502 containing cerdulatinib hydrochloride 0.1% (0.09% free base), 0.2% (0.18% free base) or 0.4 (0.37% free base) gel or vehicle gel (study medication).

Screening, Baseline, and Vehicle-Control Treatment Phases

During the Optional Active Treatment Phase, the subjects will have the option to continue participation in the study. Those subjects that elect to continue and were assigned to vehicle in the Vehicle-Control Phase will be randomized 1:1:1 in a blinded manner, DMVT-502 0.1% (0.09% free base), 0.2% (0.18% free base) or 0.4% (0.37% free base) topical gel (study medication). Those subjects that elect to continue and were assigned to one of the three active IP treatment arms in the Vehicle-Control Phase will continue to receive the same concentration.

Optional Active Treatment Phase and Follow-Up Phases

### 1.1. Treatment Arms and Duration

During the Vehicle-Control Treatment Phase, subjects will be randomized in equal numbers to one of 4 treatment arms:
Topical DMVT-502 0.1% (0.09% free base) gel BID×6 Weeks (55 subjects)
Topical DMVT-502 0.2% (0.18% free base) gel BID×6 Weeks (55 subjects)
Topical DMVT-502 0.4% (0.37% free base) gel BID×6 Weeks (55 subjects)
Topical Vehicle gel BID×6 Weeks (55 subjects)

During the Optional Active Treatment Phase, subjects assigned to vehicle from the Vehicle-Control Phase will be re-randomized in equal numbers to 1 of the 3 active treatment arms:
Topical DMVT-502 0.1% (0.09% free base) gel BID×6 Weeks
Topical DMVT-502 0.2% (0.18% free base) gel BID×6 Weeks
Topical DMVT-502 0.4% (0.37% free base) gel BID×6 Weeks

Approximately 220 subjects from approximately 60 centers in USA, Canada, and Australia will be enrolled and randomized 1:1:1:1 across the 4 treatment arms in the Double-Blind Treatment Phase of the study. The primary objective of this study is to assess the efficacy of topical DMVT-502 (cerdulatinib) gel in adult and adolescent subjects with mild, moderate, or severe atopic dermatitis. The secondary objectives of this study is to evaluate the safety, tolerability, and pharmacokinetics of topical DMVT-502 (cerdulatinib) gel in adult and adolescent subjects with atopic dermatitis.

To determine subject eligibility at Screening and Baseline, a single repeat of tests or procedures may be allowed at the discretion of the Principal Investigator in consultation with the medical monitor.

### Inclusion Criteria

A subject will be eligible for inclusion in this study when all of the following criteria apply:
1. Male and female subjects ages 12 to 60 years with confirmed diagnosis of atopic dermatitis by Hanifin and Rajka criteria [Hanifin, 1980].
For adult subjects, the age range is 18 to 60 years. For adolescent subjects, the age range is 12 to 17 years.
2. Subjects with atopic dermatitis covering ≥3% of the body surface area and with an Investigator Global Assessment (IGA) of ≥2 at screening and baseline. Scalp, palms and soles should be excluded from the BSA calculation to determine eligibility during Screening and at Baseline.
Note: Subjects with mild disease (IGA=2) and severe disease (IGA=4) will be limited to approximately 15% each of total enrollment.
3. Females of childbearing potential and male subjects who are engaging in sexual activity that could lead to pregnancy must use the following adequate birth control methods while on study and for 2 weeks after stopping study drug. Acceptable contraception methods are:
   Male partner with vasectomy, OR
   Male condom AND partner use of one of the contraceptive options below:
      Spermicide;
      Contraceptive subdermal implant that meets effectiveness criteria including a <1% rate of failure per year, as stated in the product label;
      Intrauterine device or intrauterine system that meets effectiveness criteria including a <1% rate of failure per year, as stated in the product label; o Oral contraceptive, either combined or progestogen alone;
      Injectable progestogen;
      Contraceptive vaginal ring;
      Percutaneous contraceptive patches.

Note: Subjects using hormonal contraceptives must have been on a stable dose for at least 4 weeks before baseline.

These allowed methods of contraception are only effective when used consistently, correctly and in accordance with the product label. The Investigator is responsible for ensuring that subjects understand how to properly use these methods of contraception.

Non-child-bearing potential is defined as premenarchal or pre-menopausal females with a documented bilateral tubal ligation, bilateral oophorectomy (removal of the ovaries) or hysterectomy, or hysteroscopic sterilization; or postmenopausal females defined as a cessation of menses for at least 12 months without an alternative medical cause. In questionable cases a blood sample with simultaneous follicle stimulating hormone (FSH) >40m1U is confirmatory. Documented verbal history from the subject is acceptable.

Subjects who are abstinent are eligible, but they must agree to use one of the birth control methods listed above if they start engaging in sexual activity that could lead to pregnancy during the study.

Female subjects of childbearing potential must have a negative pregnancy test at Screening and Baseline (Day 1).
4. Atopic dermatitis present for at least 12 months according to the subject and stable disease for at least 1 month according to the subject.
5. Subject, subject's parent(s), or legal representative must be capable of giving written informed consent or verbal assent, as applicable, which includes compliance with the requirements and restrictions listed in the consent/assent form; written informed consent must be obtained prior to any study related procedures.

### Exclusion Criteria

If any of the following criteria apply, then a subject is excluded and considered ineligible for continuation in this study:
1. A positive Hepatitis B surface antigen (HBsAg) or positive Hepatitis C antibody result, or a positive anti-HBc result, or medical history of positive human immunodeficiency virus (HIV) antibody at Screening.
2. Screening alanine aminotransferase (ALT) or aspartate aminotransferase (AST) ≥1.5×the upper limit of normal (ULN).
3. Screening total bilirubin >1.5×ULN; total bilirubin >ULN and ≤1.5×ULN is acceptable if bilirubin is fractionated and direct bilirubin <35%.
4. Corrected QT (QTc) interval >475 msec or >525 msec in the presence of bundle branch block.
5. Subjects with a skin condition such as Kaposi's varicelliform eruption, scabies, molluscum contagiosum, impetigo, psoriasis, severe acne, connective tissue disorder, or Netherton's syndrome, or any other disease that could impact study evaluations.
6. Use of any prohibited medication.
Prohibited concomitant medications, therapy, etc. during the defined period are as listed in the bullets below. If a subject requires any of these medications throughout the study period, he/she may be excluded from or discontinued from the study, at the discretion of the Investigator and medical monitor.
From 6 months prior to Baseline/Day 1 until the completion of the Follow-up visit or study discontinuation:
   Biological products that might have significantly affected the evaluation of atopic dermatitis condition (e.g., tumor necrosis factor [TNF] inhibitors, anti-immunoglobulin [Ig]E antibodies, anti-CD20 antibodies, anti-interleukin [IL]-4 receptor).
   From 28 days prior to Baseline/Day 1 until the completion of the Follow-up visit or discontinuation:
      EUCRISA™ (crisaborole) and any other PDE4 inhibitor;
      Corticosteroid preparations (oral, injection, and suppository preparations) and topical corticosteroids that were classified as super-high potency (eg, clobetasol propionate). Eye drops and nasal preparations are allowed. Inhaled preparations are allowed when used for a stable condition and stable dose for >28 days before Screening and are continued at the same dose throughout the study.
      Oral preparations and injections of immunosuppressants (cyclosporine, methotrexate, azathioprine, tacrolimus, etc.);
      Over the counter or herbal medicines for atopic dermatitis (topical and oral preparations);
      Excessive sun exposure, tanning booth, other ultraviolet (UV) light source and phototherapy including psoralen and ultraviolet A (PUVA) therapy or is unwilling to minimize natural and artificial sunlight exposure.
   From 14 days prior to Baseline/Day 1 until the completion of the Follow-up visit or discontinuation:
      Herbal medicines for atopic dermatitis (topical and oral preparations), unless specifically approved by the sponsor;
      Tacrolimus and pimecrolimus cream and/or ointment;
      Topical corticosteroids that were classified as low, medium, or high potency (e.g., fluocinonide, triamcinolone acetonide, desonide, hydrocortisone). Eye drops and nasal preparations are allowed.
   From 7 days prior to Baseline/Day 1 until the completion of the Follow-up visit or discontinuation:
      Oral or intravenous antibiotics, antifungal or antivirus medications
      Doxepin, topical products containing urea
      Antihistamines/anti-allergics (oral, topical and injections): diphenhydramine, chlorpheniramine maleate, hydroxyzine).
   NOTE: The following antihistamines are allowed from baseline throughout the treatment period:
      Loratadine, fexofenadine hydrochloride, cetirizine hydrochloride.
7. Pregnant females as determined by positive serum (screening) or urine (baseline) human chorionic gonadotropin test at screening or prior to dosing.
8. Lactating females.
9. History of sensitivity to the study medications, or components thereof or a history of drug or other allergy that, in the opinion of the Investigator or Medical Monitor, contraindicates their participation.
10. The subject has received an investigational product within the following time period prior to the first dosing day in the current study: 30 days, 5 half-lives or twice the duration of the biological effect of the investigational product (whichever is longer).
11. Current or a history of cancer within 5 years except for fully excised skin basal cell carcinoma, squamous cell carcinoma or carcinoma in situ of the cervix.
12. Subjects with active infection that required oral or intravenous administration of antibiotics, antifungal or antiviral agents within 7 days of Baseline/Day 1.
13. Concurrent skin lesions in the treatment area or pruritus due to conditions other than atopic dermatitis that, in the opinion of the investigator, would either interfere with study evaluations or affect the safety of the subject.
14. Subjects with advanced disease or abnormal laboratory test values that could affect the safety of the subject or the implementation of this study.
15. Previous exposure to DMVT-502.
16. History of and/or concurrent condition of serious hypersensitivity (anaphylactic shock or anaphylactoid reaction) to either JAK or SYK inhibitors.
17. Evidence of significant hepatic, renal, respiratory, endocrine, hematologic, neurologic, psychiatric, or cardiovascular system abnormalities or laboratory abnormality that will affect the health of the subject or interfere with interpretation of the results.
18. To assess any potential impact on subject eligibility with regard to safety, the Investigator must refer to the current version of the DMVT-502 Investigator Brochure for detailed information regarding warnings, precautions, contraindications, adverse events, and other significant data pertaining to the investigational product being used in this study.

### DMVT-502-2001 Study Treatments

| | Study Treatment | | | |
|---|---|---|---|---|
| | | DMVT-502 Gel Formulation B | | |
| Product name: | Vehicle | DMVT-502 | DMVT-502 | DMVT-502 |
| Study Treatment | Vehicle | 0.09% | 0.18% | 0.37% |
| Formulation | PEG-based | PEG-based | PEG-based | PEG-based |
| description: | formulation | formulation | formulation | formulation |
| Physical | Clear, slightly | Clear, slightly | Clear, slightly | Clear, slightly |
| description: | yellow to yellow | yellow to yellow | yellow to yellow | yellow to yellow |
| | gel | gel | gel | gel |
| Dosage form: | Topical gel | Topical gel | Topical gel | Topical gel |
| Unit dose | Vehicle gel/60 g | 0.09% gel/60 g | 0.18% gel/60 g | 0.37% gel/60 g |
| strength(s)/ | tubes | tubes | tubes | tubes |
| Dosage level(s): | | | | |
| Route of | Vehicle-Control | Vehicle-Control | Vehicle-Control | Vehicle-Control |
| Administration/ | Phase | Phase | Phase | Phase |
| Duration | Topical/6 weeks | Topical/6 weeks | Topical/6 weeks | Topical/6 weeks |
| | Optional Active | Optional Active | Optional Active | Optional Active |
| | Treatment Phase | Treatment Phase | Treatment Phase | Treatment Phase |
| | Topical//6 weeks | Topical/6 weeks | Topical/6 weeks | Topical/6 weeks |
| Dosing | Apply twice daily | Apply twice daily | Apply twice daily | Apply twice daily |
| instructions: | as directed | as directed | as directed | as directed |
| Manufacturer/ | Tergus Pharma, | Tergus Pharma, | Tergus Pharma, | Tergus Pharma, |
| Source of | LLC Durham, NC | LLC Durham, NC | LLC Durham, NC | LLC Durham, NC |
| Procurement | 27713 USA | 27713 USA | 27713 USA | 27713 USA |

### Atopic Dermatitis Assessments

Efficacy measurement outcomes will include:

### Eczema Area Severity Index (EASI)

The EASI will be assessed at every study visit. It quantifies the severity of a subject's atopic dermatitis based on both lesion severity and the percent of body surface area affected [Tofte, 1998]. The EASI is a composite score ranging from 0-72 that takes into account the degree of erythema, induration/papulation, excoriation, and lichenification (each scored from 0 to 3 separately) for each of four body regions, with adjustment for the percent of BSA involved for each body region and for the proportion of the body region relative to the whole body. A detailed procedure of EASI score calculation is provided in below.

Four anatomic sites—head, upper extremities, trunk, and lower extremities—are assessed for erythema, induration (papules), excoriation and lichenification as seen on the day of the examination. The severity of each sign is assessed using a 4-point scale:

0=No symptoms

### 1=Slight or Mild

### 2=Moderate

### 3=Marked or Severe

The area affected by atopic dermatitis within a given anatomic site is estimated as a percentage of the total area of that anatomic site and assigned a numerical value according to the degree of atopic dermatitis involvement as follows:

0=no involvement 1=<10%
2=10 to <30%
3=30 to <50%
4=50 to <70% 5=70 to <90% 6=90 to 100%
The EASI score is obtained by using the formula:

EASI=0.1 (Eₕ+Iₕ+Exₕ+Lₕ) Aₕ+0.2 (Eᵤ+Iᵤ+Exᵤ+Lᵤ) Aᵤ+0.3 (Eₜ+Iₜ+Exₜ+Lₜ) Aₜ+0.4 (Eₗ+Iₗ+Exₗ+Lₗ) Aₗ

Where E, I, Ex, L and A denote erythema, induration, excoriation, lichenification and area, respectively, and h, u, t, and l denote head, upper extremities, trunk, and lower extremities, respectively.

### Investigator Global Assessment (IGA)

The IGA of disease severity will be assessed at every clinic visit. The IGA is a global assessment of the current state of the disease. It is a 5-point morphological assessment of overall disease severity and will be determined according to the categories described in the table below. To be eligible, subjects must have an IGA score of 2 or greater at the Screening and Baseline visit (Day 1).

### Investigator Global Assessment Categories of Disease Severity

| Score | Category | Definition |
|---|---|---|
| 0 | Clear | Minor, residual discoloration, no erythema or |
| | | induration/papulation, no oozing/crusting |
| 1 | Almost | Trace, faint pink erythema with almost no |
| | clear | induration/papulation and no oozing/crusting |
| 2 | Mild | Faint pink erythema with mild induration/ |
| | disease | papulation and no oozing/crusting |
| 3 | Moderate | Pink-red erythema with moderate induration/ |
| | disease | papulation and there may be some oozing/crusting |
| 4 | Severe | Deep/bright red erythema with severe induration/ |
| | disease | papulation with oozing/crusting |

### EXAMPLE 8

### Study DMVT-502-9025: Cerdulatinib (DMVT-502) Treatment in a Mouse Model of Vitiligo

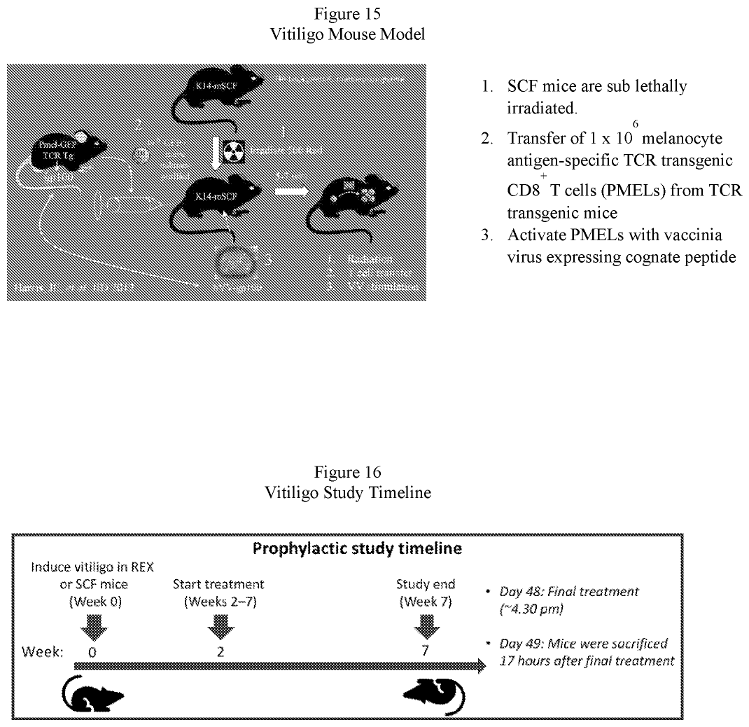

Cerdulatinib was evaluated in a vitiligo mouse model (Harris J E et al. J Invest Dermatol. 2012;132:1869-1876). The model mimics human vitiligo by inducing epidermal depigmentation with autoreactive CD8+ T-cell accumulation. A schematic of the model is depicted in FIG. 15.

After vitiligo was induced in mice, the mice were treated with cerdulatinib (30 or 60 mg/kg) or vehicle via once daily oral administration for 5 weeks. Ten mice were used in the study: three REX3 reporter mice for cytokines CXCL9 and CXCL10 and seven SCF (stem cell factor) mice. The vitiligo study timeline is depicted in FIG. 16. The following assessments were performed during the study:
Blinded scoring of ears, tail, nose, and ventral feet (0-5 scale)
Epidermal, dermal, spleen, and lymph node PMEL (premelanosome protein) cell count
Epidermal/dermal APC (antigen-presenting cell) count
Keratinocyte cytokine expression

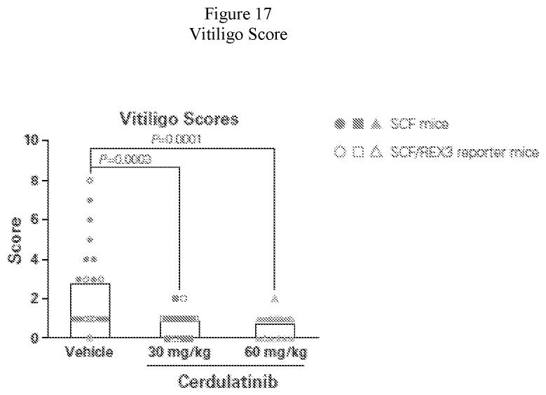

The vitiligo scores obtained during the study are depicted in FIG. 17. Cerdulatinib 30 and 60 mg/kg significantly decreased vitiligo scores compared with vehicle controls.

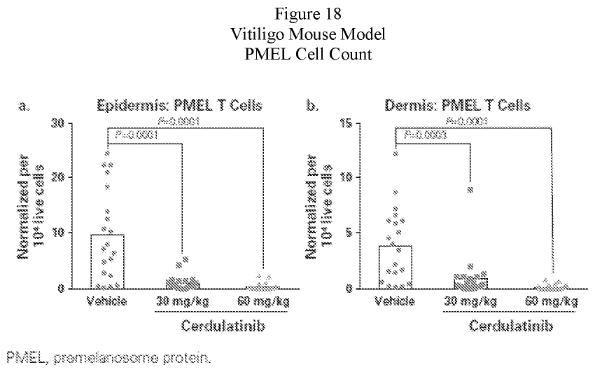

The PMEL cell count measurements are depicted in FIG. 18. Cerdulatinib 30 and 60 mg/kg significantly reduced PMEL T-cell counts in the epidermis and dermis compared with vehicle. In addition, cerdulatinib 60 mg/kg significantly reduced PMEL T-cell counts in dermis and blood. There were no significant differences in lymph node or spleen PMEL T cells.

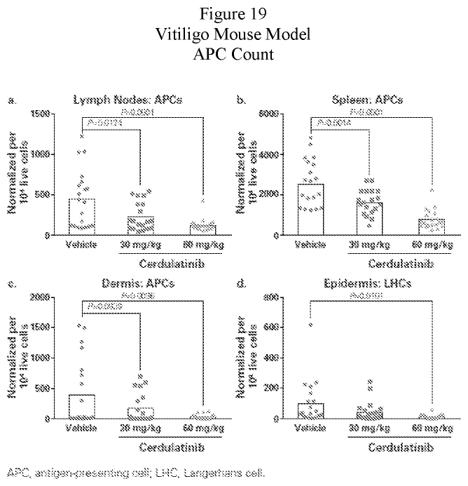

The APC count measurements are depicted in FIG. 19. Cerdulatinib 30 and 60 mg/kg significantly reduced APC counts in lymph nodes and the spleen compared with vehicle. Reduced CXCL9 and CXCL10 expression in lymph node APCs was observed. In the dermis and Langerhans cells, reduced APC counts were recorded, with reduced CXCL10 expression. Reduced CD3+ CD8− T cells were also observed. Cerdulatinib 60 mg/kg significantly reduced APCs in the dermal skin compartment and Langerhans cells in the epidermal skin compartment compared with vehicle.

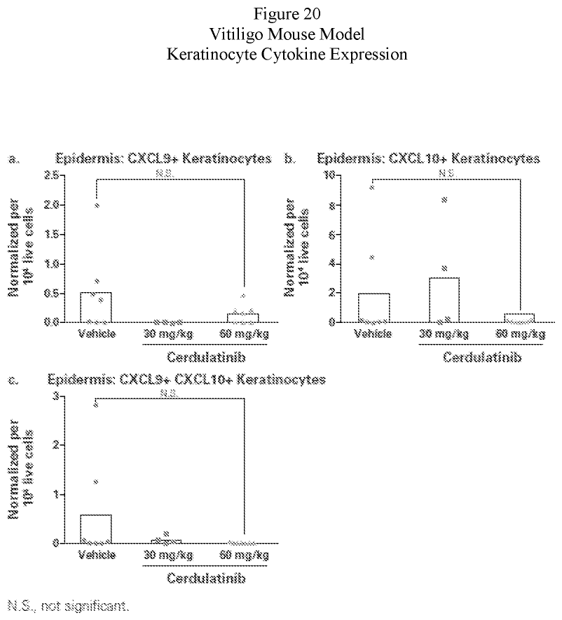

The keratinocyte cytokine expression is depicted in FIG. 20. Cerdulatinib treatment in SCF/REX3 mice resulted in trends towards reduced CXCL9, CXCL10, and dual expression in keratinocytes compared with vehicle. No decrease in total keratinocyte counts between treatment groups were observed.

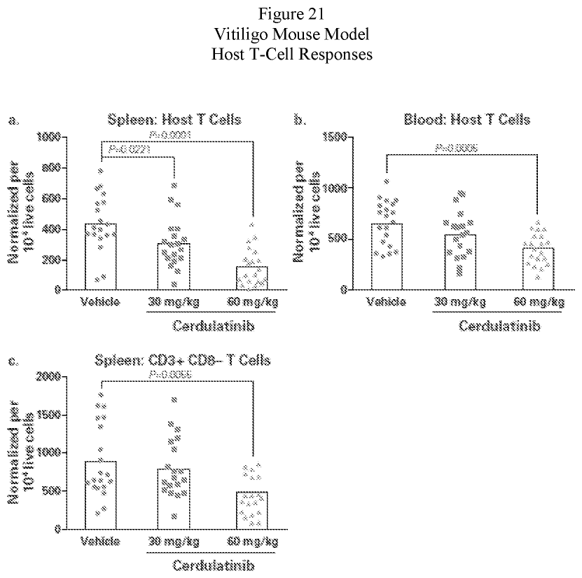

As seen in FIG. 21, cerdulatinib 30 and 60 mg/kg significantly reduced host-derived total T-cell counts in the spleen. Host T-cell counts in blood were significantly decreased in mice treated with cerdulatinib 60 mg/kg. CD3+, CD8− T-cell counts, indicative of CD4+ T-cell population in the spleen were significantly reduced in response to cerdulatinib 60 mg/kg.

In summary, the vitiligo study revealed a significant decrease in vitiligo score with both cerdulatinib treatment groups. In addition, a significant decrease in PMEL T-cell numbers in both epidermis and dermis was observed, as well as trends for reduction in APCs in skin tissues. The study also showed a general trend for reduction of chemokine expression in skin cells (Langerhans, dermal APCs, and keratinocytes).

All literature and patent references cited throughout the application are incorporated into the application by reference for all purposes.

In view of the many possible embodiments to which the principles of the disclosed invention may be applied, it should be recognized that the illustrated embodiments are the only preferred examples of the invention and should not be taken as limiting the scope of the invention. Rather, the scope of the invention is defined by the following claims. We therefore claim as our invention all that comes within the scope and spirit of these claims.

### References Cited

Atherton D J. Topical corticosteroids in atopic dermatitis. BMJ. 2003 Oct. 25;327(7421):942-3.
Ballardini N, Kull I, Soderhall C, Lilja G, Wickman M, Wahlgren C F. Eczema severity in preadolescent children and its relation to sex, filaggrin mutations, asthma, rhinitis, aggravating factors and topical treatment: a report from the BAMSE birth cohort. Br J Dermatol. 2013 March;168(3):588-94.
Bieber T. Atopic dermatitis. N Engl J Med. 2008 Apr. 3;358(14):1483-94.
Boguniewicz M, Leung D Y. Atopic dermatitis: a disease of altered skin barrier and immune dysregulation. Immunol Rev. 2011 July;242(1):233-46.
Choi O H, Kim J H, Kinet J P. Calcium mobilization via sphingosine kinase in signalling by the Fc epsilon RI antigen receptor. Nature. 1996 Apr. 18;380(6575):634-6.
Dalgard F J, Gieler U, Tomas-Aragones L, Lien L, Poot F, Jemec G B E, Misery L, Szabo C, Linder D, Sampogna F, Evers A W M, Halvorsen J A, Balieva F, Szepietowski J, Romanov D, Marron S E, Altunay I K, Finlay A Y, Salek S S, Kupfer J. The psychological burden of skin diseases: a cross-sectional multicenter study among dermatological out-patients in 13 European countries. J Invest Dermatol. 2015 April;135(4):984-991.
Darby C, Geahlen R L, Schreiber A D. Stimulation of macrophage Fc gamma RIIIA activates the receptor-associated protein tyrosine kinase Syk and induces phosphorylation of multiple proteins including p95Vav and p62/GAP-associated protein. J Immunol. 1994 Jun. 1;152(11):5429-37.

Deckers I A, McLean S, Linssen S, Mommers M, van Schayck C P, Sheikh A. Investigating international time trends in the incidence and prevalence of atopic eczema 1990-2010: a systematic review of epidemiological studies. PLoS One. 2012;7(7):e39803.

Elidel Prescribing Information. Revised June 2017. Available at: http://elidel-us.com/hcp/. Accessed: 13 Oct. 2017.
Evans D C, Watt A P, Nicoll-Griffith D A, Baillie T A. Drug-protein adducts: an industry perspective on minimizing the potential for drug bioactivation in drug discovery and development. Chem Res Toxicol. 2004 January;17(1):3-16.
Garmhausen D, Hagemann T, Bieber T, Dimitriou I, Fimmers R, Diepgen T, Novak N. Characterization of different courses of atopic dermatitis in adolescent and adult patients. Allergy. 2013 April;68(4):498-506.

Hanifin J M, Rajka G. Diagnostic features of atopic dermatitis. Acta Dermato-Venereologica Supplementum. 1980;92:44-7.

Harris J E et al. J Invest Dermatol. 2012;132:1869-1876.
Illi S, von Mutius E, Lau S, Nickel R, Gruber C, Niggemann B, Wahn U; Multicenter Allergy Study Group. The natural course of atopic dermatitis from birth to age 7 years and the association with asthma. J Allergy Clin Immunol. 2004 May;113(5):925-31.
Kuo I H, Yoshida T, De Benedetto A, Beck L A. The cutaneous innate immune response in patients with atopic dermatitis. J Allergy Clin Immunol. 2013 February;131(2):266-78.
O'Shea J J, Pesu M, Borie D C, Changelian P S. A new modality for immunosuppression: targeting the JAK/STAT pathway. Nat Rev Drug Discov. 2004 July;3(7):555-64.
Pastore S, Mascia F, Girolomoni G. The contribution of keratinocytes to the pathogenesis of atopic dermatitis. Eur J Dermatol. 2006 March-April;16(2):125-31.
Pesu M, Laurence A, Kishore N, Zwillich S H, Chan G, O'Shea J J. Therapeutic targeting of Janus kinases. Immunol Rev. 2008 June;223:132-42.
Protopic Prescribing Information. Revised May 2012. Available at: https://astellas.us/docs/protopic.pdf. Accessed: 13 Oct. 2017.
Silverberg J I, Hanifin J M. Adult eczema prevalence and associations with asthma and other health and demographic factors: a US population-based study. J Allergy Clin Immunol. 2013 November;132(5):1132-8.
Smith-Garvin J E, Koretzky G A, Jordan M S. T cell activation. Annu Rev Immunol. 2009;27:591-619.
Tofte S, Graeber M, Cherill R, Omoto M, Thurston M, Hanifin J M. Posters—P48—Eczema area and severity index (EAST): A new tool to evaluate atopic dermatitis. [abstract]. J Eur Acad Dermatol Venereol. 1998;11(suppl 2):S197.
Werfel T, Allam J P, Biedermann T, Eyerich K, Gilles S, Guttman-Yassky E, Hoetzenecker W, Knol E, Simon H U, Wollenberg A, Bieber T, Lauener R, Schmid-Grendelmeier P, Traidl-Hoffmann C, Akdis C A. Cellular and molecular immunologic mechanisms in patients with atopic dermatitis. J Allergy Clin Immunol. 2016 August;138(2):336-49.
Williams H, Flohr C. How epidemiology has challenged 3 prevailing concepts about atopic dermatitis. J Allergy Clin Immunol. 2006 July;118(1):209-13.
Williams H, Stewart A, von Mutius E, Cookson W, Anderson H R, International Study of Asthma and Allergies in Childhood (ISAAC) Phase One and Three Study Groups. Is eczema really on the increase worldwide? J Allergy Clin Immunol. 2008 April;121(4):947-54.e15.

1. A pharmaceutical composition for topical use, comprising:
a therapeutically effective amount of cerdulatinib or a pharmaceutically acceptable salt, hydrate or solvate thereof;
a pharmaceutically acceptable carrier comprising a polyalkylene glycol having an average molecular weight of from 100 daltons to 10,000 daltons and
propylene glycol.

2. The pharmaceutical composition according to claim 1, wherein the cerdulatinib or a pharmaceutically acceptable salt, hydrate or solvate thereof, is cerdulatinib hydrochloride.

3. The pharmaceutical composition according to claim 1, wherein the polyalkylene glycol is selected from the group consisting of a polyethylene glycol, a polyethylene glycol having an average molecular weight of 100 daltons to 5,000 daltons, a polyethylene glycol having an average molecular weight of 200 daltons to 600 daltons, a polyethylene glycol having an average molecular weight of 2,000 daltons to 6,000 daltons, a polyethylene glycol having an average molecular weight of 1,000 daltons to 10,000 daltons, PEG 400, and PEG 4000.

4-6. (canceled)

7. The pharmaceutical composition according to claim 1, further comprising one or more component selected from the group consisting of a penetration enhancer, glycerol, hydroxypropyl, cellulose, an antimicrobial preservative, an antioxidant, and combinations thereof.

8-14. (canceled)

15. The pharmaceutical composition according to claim 7, wherein the antioxidant comprises butylated hydroxytoluene.

16. The pharmaceutical composition according to claim 7, wherein the penetration enhancer comprises Transcutol HP.

17. The pharmaceutical composition according to claim 7, wherein the antimicrobial preservative comprises phenoxyethanol.

18. The pharmaceutical composition according to claim 1, wherein the therapeutically effective amount of cerdulatinib, or a pharmaceutically acceptable salt, hydrate or solvate thereof, is selected from the group consisting of about 0.01% to about 5.0% (w/w), about 0.05% to about 3.0% (w/w), about 0.05% to about 1.0% (w/w), about 0.075% to about 0.75% (w/w), about 0.1% (w/w), about 0.2% (w/w), and about 0.4% (w/w).

19-21. (canceled)

22. The pharmaceutical composition according to claim 1, wherein the pharmaceutical composition is a gel or an ointment.

23-27. (canceled)

28. A pharmaceutical composition for topical use, comprising:
0.01-5.0% (w/w) of cerdulatinib or a pharmaceutically acceptable salt, hydrate or solvate thereof;
30-70% (w/w) of a polyethylene glycol with an average molecular weight of 200 daltons to 600 daltons;
5.0-25% (w/w) of propylene glycol; and
5.0-50% (w/w) of a penetration enhancer.

29. The pharmaceutical composition of claim 28, comprising:
0.05 to 3.0% (w/w) of cerdulatinib or a pharmaceutically acceptable salt, hydrate or solvate thereof hydrochloride;
35-65% (w/w) of a polyethylene glycol with an average molecular weight of 200 daltons to 600 daltons;
10-20% (w/w) of propylene glycol; and
5.0-25% (w/w) of a penetration enhancer.

30. The pharmaceutical composition of claim 28, comprising:
0.05 to 1.0% (w/w) of cerdulatinib or a pharmaceutically acceptable salt, hydrate or solvate thereof hydrochloride;
40-55% (w/w) of a polyethylene glycol with an average molecular weight of 200 daltons to 600 daltons;
10-20% (w/w) of propylene glycol; and
10-20% (w/w) of a penetration enhancer.

31. The pharmaceutical composition according to claim 7, wherein the pharmaceutical composition further comprises a component and the component is hydroxypropyl cellulose in an amount of about 0.1% to about 3.0% (w/w) or about 0.5% to about 2.0% (w/w).

32. The pharmaceutical composition according to claim 7, wherein the pharmaceutical composition further comprises a component and the component is glycerol in an amount of about 10% to about 35% (w/w) or about 20% to about 30% (w/w).

33-34. (canceled)

35. The pharmaceutical composition of claim 7, wherein the pharmaceutical composition further comprises a component and the component is an antioxidant in an amount of about 0.01% to about 1.0% (w/w) and an antimicrobial preservative in an amount of about 0.01% to about 2.0% (w/w).

36-37. (canceled)

38. The pharmaceutical composition according to claim 3, wherein the polyethylene glycol is polyethylene glycol with an average molecular weight of 2,000 daltons to 6,000 daltons and is present in an amount of about 15% to about 30%.

39-45. (canceled)

46. A method for treating a dermatological disorder comprising topically administering a pharmaceutical composition having the formulation of claim 1.

47. (canceled)

48. The method according to claim 46, wherein the dermatological disorder is selected from the group consisting of alopecia areata, chronic urticaria, vitiligo, atopic dermatitis, moderate atopic dermatitis, and severe atopic dermatitis.

49-56. (canceled)

57. The method of claim 46, wherein the formulation is applied once or twice daily.

58. The method of claim 46, wherein the formulation is a gel or an ointment.

59. (canceled)

60. The method of claim 58, wherein the flux of cerdulatinib from the gel is greater than 0.2 ng/cm²/hr as determined using a MedFlux-HT™ diffusion cell or the flux of cerdulatinib from the ointment is greater than 0.06 ng/cm²/hr as determined using a MedFlux-HT™ diffusion cell.

61. (canceled)
